SEQUENCE LISTING (16 sequences)

SEQ ID NO: 1 - protein, 222 aa, Artificial Sequence
  Influenza H1 PR8-H1N1 Hemagglutinin
GLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 2 - protein, 222 aa, Artificial Sequence
  H2 (L11136) Hemagluttinin
GLFGAIAGFIEGGWQGMIDGWYGYHHSNDQGSGYAADKESTQKAIDGITNRVNSVIEKMNTQFEAVGKEFSNLEKRLENLNKKMEDGFLDVWTYNAELLVLMENERTLDFHDSNVKNLYDRVRMQLRDNAKELGNGCFEFYHKCDDECMNSVKNGTYDYPKYEEESKLNRNEIKGVKLSNMGVYQILAIYATVAGSLSLAIMIAGISLWMCSNGSLQCRICI

SEQ ID NO: 3 - protein, 221 aa, Artificial Sequence
  H3 HK68-H3N2 (EF4090245) PDB1HGJ Hemagglutinin
GLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLPEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 4 - protein, 221 aa, Artificial Sequence
  H4 (D90302) Hemagglutinin
GLFGAIAGFIENGWQGLIDGWYGFRHQNAEGTGTAADLKSTQAAIDQINGKLNRLIEKTNDKYHQIEKEFEQVEGRIQDLENYVEDTKIDLWSYNAELLVALENQHTIDVTDSEMNKLFERVRRQLRENAEDKGNGCFEIFHKCDNNCIESIRNGTYDHDIYRDEAINNRFQIQGVKLTQGYKDIILWISFSISCFLLVALLLAFILWACQNGNIRCQICI

SEQ ID NO: 5 - protein, 222 aa, Artificial Sequence
  H5 (X07826) Hemagglutinin
GLFGAIAGFIEGGWQGMVDGWYGYHHSNEQGSGYAADKESTQKAIDGITNKVNSIIDKMNTRFEAVGKEFNNLERRVENLNKKMEDGFLDVWTYNVELLVLMENERTLDFHDSNVNNLYDKVRLQLKDNARELGNGCFEFYHKCDNECMESVRNGTYDYPQYSEEARLNREEISGVKLESMGVYQILSIYSTVASSLALAIMIAGLSFWMCSNGSLQCRICI

SEQ ID NO: 6 - protein, 222 aa, Artificial Sequence
  H6 (D90303) Hemagglutinin
GLFGAIAGFIEGGWTGMIDGWYGYHHENSQGSGYAADRESTQKAVDGITNKVNSIIDKMNTQFEAVDHEFSNLERRIDNLNKRMEDGFLDVWTYNAELLVLLENERTLDLHDANVKNLYERVKSQLRDNAMILGNGCFEFWHKCDDECMESVKNGTYDYPKYQDESKLNRQEIESVKLESLGVYQILAIYSTVSSSLVLVGLIIAVGLWMCSNGSMQCRICI

SEQ ID NO: 7 - protein, 221 aa, Artificial Sequence
  H7 (M24457) Hemagglutinin
GLFGAIAGFIENGWEGLVDGWYGFRHQNAQGEGTAADYKSTQSAIDQITGKLNRLIEKTNQQFELIDNEFTEVEKQIGNLINWTKDSITEVWSYNAELIVAMENQHTIDLADSEMNRLYERVRKQLRENAEEDGTGCFEIFHKCDDDCMASIRNNTYDHSKYREEAMQNRIQIDPVKLSSGYKDVILWFSFGASCFLLLAIAMGLVFICVKNGNMRCTICI

SEQ ID NO: 8 - protein, 222 aa, Artificial Sequence
  H8 (D90304) Hemagglutinin
GLFGAIAGFIEGGWSGMIDGWYGFHHSNSEGTGMAADQKSTQEAIDKITNKVNNIVDKMNREFEVVNHEFSEVEKRINMINDKIDDQIEDLWAYNAELLVLLENQKTLDEHDSNVKNLFDEVKRRLSANAIDAGNGCFDILHKCDNECMETIKNGTYDHKEYEEEAKLERSKINGVKLEENTTYKILSIYSTVAASLCLAILIAGGLILGMQNGSCRCMFCI

SEQ ID NO: 9 - protein, 222 aa, Artificial Sequence
  H9 (D90305) Hemagglutinin
GLFGAIAGFIEGGWPGLVAGWYGFQHSNDQGVGMAADKGSTQKAIDKITSKVNNIIDKMNKQYEVIDHEFNELEARLNMINNKIDDQIQDIWAYNAELLVLLENQKTLDEHDANVNNLYNKVKRALGSNAVEDGNGCFELYHKCDDQCMETIRNGTYDRQKYQEESRLERQKIEGVKLESEGTYKILTIYSTVASSLVLAMGFAAFLFWAMSNGSCRCNICI

SEQ ID NO: 10 - protein, 221 aa, Artificial Sequence
  H10 (M21647) Hemagglutinin
GLFGAIAGFIENGWEGMVDGWYGFRHQNAQGTGQAADYKSTQAAIDQITGKLNRLIEKTNTEFESIESEFSETEHQIGNVINWTKDSITDIWTYNAELLVAMENQHTIDMADSEMLNLYERVRKQLRQNAEEDGKGCFEIYHTCDDSCMESIRNNTYDHSQYREEALLNRLNINPVKLSSGYKDIILWFSFGESCFVLLAVVMGLCFFCLKNGNMRCTICI

SEQ ID NO: 11 - protein, 223 aa, Artificial Sequence
  H11 (D90306) Hemagglutinin
GLFGAIAGFIEGGWPGLINGWYGFQHRDEEGTGIAADKESTQKAIDQITSKVNNIVDRMNTNFESVQHEFSEIEERINQLSKHVDDSVVDIWSYNAQLLVLLENEKTLDLHDSNVRNLHEKVRRMLKDNAKDEGNGCFTFYHKCDNKCIERVRNGTYDHKEFEEESKINRQEIEGVKLDSSGNVYKILSIYSCIASSLVLAALIMGFMFWACSNGSCRCTICI

SEQ ID NO: 12 - protein, 222 aa, Artificial Sequence
  H12 (D90307) Hemagglutinin
GLFGAIAGFIEGGWPGLVAGWYGFQHQNAEGTGIAADRDSTQRAIDNMQNKLNNVIDKMNKQFEVVNHEFSEVESRINMINSKIDDQITDIWAYNAELLVLLENQKTLDEHDANVRNLHDRVRRVLRENAIDTGDGCFEILHKCDNNCMDTIRNGTYNHKEYEEESKIERQKVNGVKLEENSTYKILSIYSSVASSLVLLLMIIGGFIFGCQNGNVRCTFCI

SEQ ID NO: 13 - protein, 223 aa, Artificial Sequence
  H13 (D90308) Hemagglutinin
GLFGAIAGFIEGGWPGLINGWYGFQHQNEQGTGIAADKESTQKAIDQITTKINNIIDKMNGNYDSIRGEFNQVEKRINMLADRIDDAVTDIWSYNAKLLVLLENDKTLDMHDANVKNLHEQVRRELKDNAIDEGNGCFELLHKCNDSCMETIRNGTYDHTEYAEESKLKRQEIDGIKLKSEDNVYKALSIYSCIASSVVLVGLILSFIMWACSSGNCRFNVCI

SEQ ID NO: 14 - protein, 221 aa, Artificial Sequence
  H14 (M35997) Hemagglutinin
GLFGAIAGFIENGWQGLIDGWYGFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDQGNGCFEIFHQCDNNCIESIRNGTYDHNIYRDEAINNRIKINPVTLTMGYKDIILWISFSMSCFVFVALILGFVLWACQNGNIRCQICI

SEQ ID NO: 15 - protein, 220 aa, Artificial Sequence
  H15 (L43917) Hemagglutinin
GLFGAIAGFIENGWEGLIDGWYGFRHQNAQGQGAADYKSTQAAIDQITGKLNRLIEKTNKQFELIDNEFTEVEQQIGNVINWTRDSLTEIWSYNAELLVAMENQHTIDLADSEMNKLYERVRRQLRENAEEDGTGCFEIFHRCDDQCMESIRNNTYNHTEYRQEALQNRIMINPVKLSSGYKDVILWFSFGASCVMLLAIAMGLIFMCVKNGNLRCTICI

SEQ ID NO: 16 - protein, 223 aa, Artificial Sequence
  H16 (EU293865) Hemagglutinin
GLFGAIAGFIEGGWPGLINGWYGFQHQNEQGTGIAADKASTQKAINEITTKINNIIEKMNGNYDSIRGEFNQVEKRINMLADRVDDAVTDIWSYNAKLLVLLENDTRLDLHDANVRNLHDQVKRALKSNAIDEGDGCFNLLHKCNDSCMETIRNGTYNHEDYREESQLKRQEIEGIKLKTEDNVYKVLSIYSCIASSIVLVGLILAFIMWACSNGSCRFNVCI

(19) United States
(12) Patent Application Publication
(10) Pub. No.: US 2015/0366962 A1
(43) Pub. Date: Dec. 24, 2015

(54) SYNTHETIC TLR4 AND TLR7 LIGANDS AS VACCINE ADJUVANTS

(71) Applicants: The Regents of the University of California, Oakland, CA (US); ICAHN SCHOOL OF MEDICINE AT MOUNT SINAI, New York, NY (US)

(72) Inventors: Dennis A. Carson, La Jolla, CA (US); Howard B. Cottam, Escondido, CA (US); Tomoko Hayashi, San Diego, CA (US); Maripat Corr, San Diego, CA (US); Peter Palese, Leonia, NJ (US); Peter Goff, New York, NY (US)

(21) Appl. No.: 14/745,394

(22) Filed: Jun. 20, 2015

Related U.S. Application Data
(60) Provisional application No. 62/014,796, filed on Jun. 20, 2014.

(51) Int. Cl. A61K 39/39; C12N 7/00; A61K 39/145

(52) U.S. Cl. CPC A61K 39/39; A61K 39/145; C12N 7/00; C12N 2760/16034

(57) ABSTRACT

This disclosure relates to compositions and methods useful to augment an immune response and methods and compositions for inducing immunogenicity to influenza antigens.

### CROSS REFERENCE TO RELATED APPLICATIONS

The application claims priority under 35 U.S.C. §119 to U.S. Provisional Application Ser. No. 62/014,796, filed Jun. 20, 2014, the disclosure of which is incorporated herein by reference.

### STATEMENT REGARDING FEDERALLY SPONSORED RESEARCH

The invention was made with U.S. Government Support under Grant No. HHSN272200900034C awarded by the National Institutes of Health. The Government has certain rights in the invention.

### TECHNICAL FIELD

This disclosure relates to compositions and methods useful to augment an immune response and methods and compositions for inducing immunogenicity to influenza antigens.

### BACKGROUND

Since influenza viruses are constantly undergoing change (antigenic drift), it is difficult to predict what subtype and strain of virus will be circulating in the next influenza season or pandemic, and to allow sufficient time (about 6 months) for vaccine manufacture and distribution. Thus, the effectiveness of a conventional vaccine against seasonal influenza is limited to the subtype and strain that was correctly predicted at the time of vaccine manufacture, well before the beginning of the influenza season. These conventional vaccines are typically based on antigens associated with the influenza hemagglutinin (HA) protein, and in particular, the globular head domain of the protein. This highly antigenic head domain is variable across strains and subtypes of influenza viruses and thus, an immune response against one globular head domain subtype may be limited to that particular head domain and fail to provide an adequate response against a virus strain having a different head domain. Influenza HA antigens based on the stem or stalk domain of the protein, which are more highly conserved across virus strains, are generally less immunogenic than the head domain antigens.

### SUMMARY

The disclosure provides a composition comprising a first aqueous component and a second component, wherein said second component comprises a compound of Formula (I) and a compound of formula (II), or a composition comprising a compound of Formula (I) and a composition comprising a compound of Formula (II):

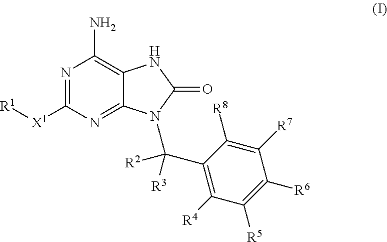

wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉)heterocyclic, substituted (C₅₋₉)heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or

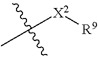

and wherein at least one of R⁴-R⁸ is

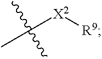

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof;

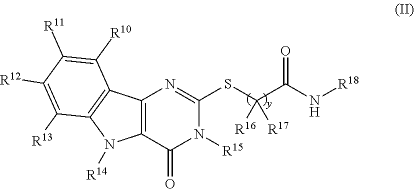

or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5; and wherein the aqueous component comprises an immunogen. In one embodiment, the immunogen is selected from the group consisting of virus, bacteria, fungus and pathogen products derived from said virus, bacteria, or fungus. In a further embodiment, the virus is selected from the group consisting of influenza A virus, avian influenza virus, H5N1 influenza virus, West Nile virus, SARS virus, Marburg virus, Arenaviruses, Nipah virus, alphaviruses, filoviruses, herpes simplex virus I, herpes simplex virus II, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus. In another embodiment, the bacteria is selected from the group consisting of Bacillus cereus, Bacillus circulans and Bacillus megaterium, Bacillus anthracia, bacterial of the genus Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis. In one embodiment, the immunogen is an influenza antigen. In a specific embodiment, the influenza antigen is an HA stalk antigen. In another embodiment, Formula I is defined to have the structure of Formula I(a):

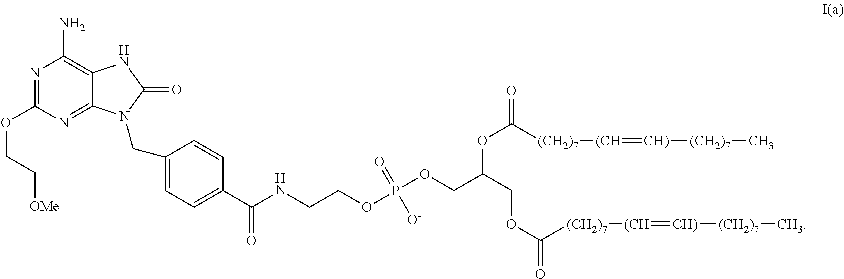

In another embodiment, Formula II is defined to have the structure of Formula II(a):

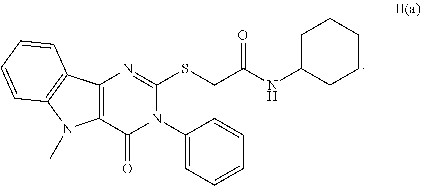 [SMILES: Cn1c2ccccc2c2nc(SCC(=O)NC3CCCCC3)n(-c3ccccc3)c(=O)c21]

In another embodiment, the first aqueous component and the second component form an emulsion. In still another embodiment, the first aqueous component and second component form a suspension.

The disclosure also provides a method to augment an immune response in a mammal comprising administering to the mammal an effective amount of any of the foregoing compositions.

The disclosure also provides a method to augment an immune response in a mammal, comprising administering to the mammal an effective amount of a composition comprising a compound of Formula (I) and a compound of formula (II), or a composition comprising a compound of Formula (I) and a composition comprising a compound of Formula (II):

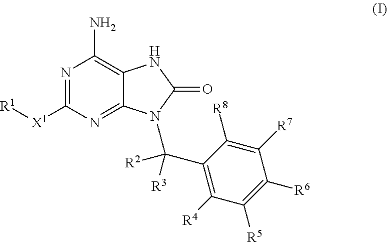

wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉)heterocyclic, substituted (C₅₋₉)heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or

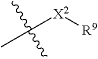

and wherein at least one of R⁴-R⁸ is

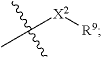

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof;

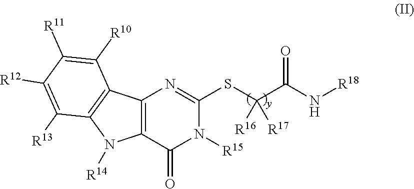

or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5. In one embodiment, the composition comprising a compound of formula (I) and (II) further comprises an amount of an immunogen. In one embodiment, the immunogen is a microbe, protein or a spore. In a further embodiment, the immunogen is an influenza HA stalk peptide. In another embodiment, the method further comprises administering an antigen. In one embodiment, the antigen is administered concurrently with the composition. In another embodiment, the antigen is administered before or after the composition. In another embodiment, the antigen is a microbe, protein or spore. In a further embodiment, the antigen is an influenza antigen. In still a further embodiment, the antigen is an influenza HA stalk peptide. In one embodiment, the composition is administered as a nanoemulsion. In still another embodiment, the composition is administered as a suspension. In another embodiment, the administration is effective to prevent, inhibit or treat a microbial infection.

The disclosure also provides a vaccine comprising a composition comprising an antigen and an amount of a compound having Formula (I) and a compound having formula (II), or a tautomer thereof, or a pharmaceutically acceptable salt or solvate thereof:

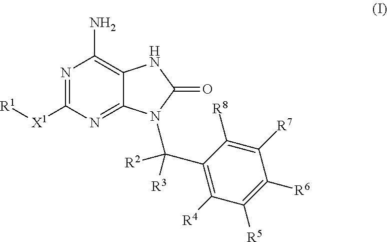

wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉) heterocyclic, substituted (C₅₋₉) heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O) NR^{a}R^{b} (carbamoyl), halo, nitro, cyano or

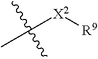

and wherein at least one of R⁴-R⁸ is

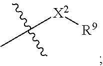

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof;

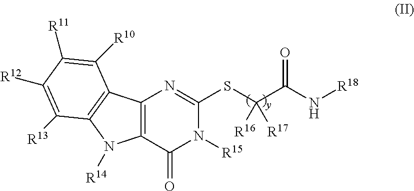

or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5. In one embodiment, Formula I is defined to have the structure of Formula I(a):

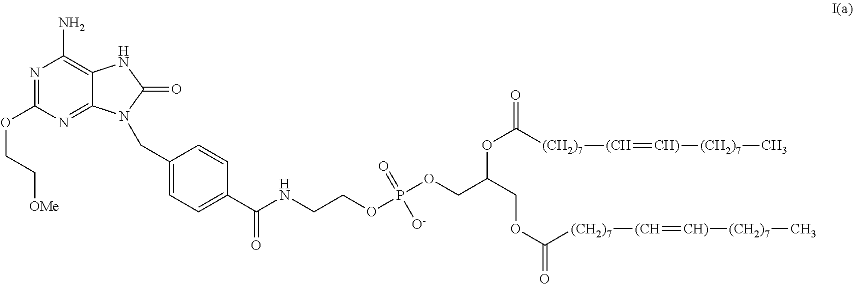

and wherein Formula II is defined to have the structure of Formula II(a):

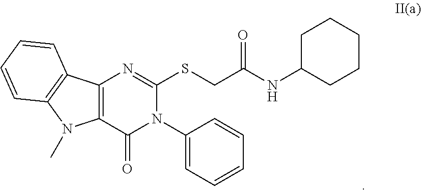 [SMILES: Cn1c2ccccc2c2nc(SCC(=O)NC3CCCCC3)n(-c3ccccc3)c(=O)c21]

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1 shows the structures of 1Z105 and IV270.

FIG. 2A-E shows that 1Z105 enhances the antigen presentation function of murine dendritic cells and is active in human dendritic cells. (A) Upregulation of antigen uptake by 1Z105. BMDCs prepared from C57BL/6 mice were incubated with 10 μM 1Z105 or MPLA overnight. Antigen, Alexa Fluor 488-conjugated ovalbumin (OVA-488), was added to the culture for the last 30 min of incubation. The cells were washed and stained for CD11c. OVA-associated dendritic cells in the CD11c^{hi} population were evaluated by flow cytometry. Cells incubated at 4° C. served as a negative control. (B and C) Enhancement of expression of costimulatory molecules by 1Z105.WT (B) or Tlr4^{-/-} (C) BMDCs were incubated with 1Z105 (10 or 2 μM) or MPLA (0.04 μg/ml) overnight, and expression of CD40 and CD86 was assessed by flow cytometric assay. (D) 1Z105 promotes antigen cross-presentation. Wild-type BMDCs were incubated with 1Z105 (2 μM) overnight, and OVA (10 μg/ml) was added to the culture for the last 4 h of incubation. The cells were washed and cultured with CFSE-labeled CD8⁺ OT1Tcells for 3 days. OT-1 T-cell proliferation was monitored by flow-cytometric assay. The data shown are representative of two independent experiments showing similar results. The flow cytometry data are representative of at least 3 independent experiments. (E) Human myeloid dendritic cells were incubated with 1Z105 (10 μM) for 18 h. Cytokine release of IL-1β, IL-6, IL-8, IL-12p70, and TNF-α was determined by Luminex bead assay. The data are the average and SEM of measurements obtained from hDCs from three independent donors. *, P 0.05, indicating a significant difference from the vehicle (Veh; 0.5% DMSO) by one-way ANOVA with Dunnett's post hoc testing.

FIG. 3A-E shows assessment of the adjuvant properties of 1Z105 using a model antigen, OVA. (A to C) C57BL/6 mice (n 8 to 16/group) were i.m. immunized with OVA (20 μg/mouse) plus 1Z105, 1V270, or a combination of 1Z105 and 1V270 in a 50-μl volume on days 0 and 14. AddaVax and vehicle were used as controls. The sera were collected on day 35, and the serum IgG1 (A) and IgG2c (B) levels were tested. (C) Splenocytes were incubated with OVA (100 μg/ml) for 3 days, and IFN-γ levels in the culture supernatants were determined by ELISA. Data were pooled from three experiments showing similar trends. (D and E) Myd88^{-/-}, Tri^{flps/lps}, or wild-type mice (n=8/group) were i.m. immunized with ovalbumin mixed with the indicated adjuvant or vehicle in a 50-μl volume on days 0 and 14. On day 35, splenocytes were incubated with OVA MHC class I peptides (OVA₂₅₇₋₂₆₄) (D) or class II peptides (OVA₃₂₃₋₃₃₉) (E) overnight, and IFN-γ-producing cells were detected by ELISpot assay. The data shown are pooled from 2 or 3 independent experiments showing similar trends. *, P 0.05 compared to no adjuvant (A to C) or the WT (D and E) by one-way ANOVA with Dunnett's post hoc testing.

FIG. 4 shows the phylogenetic relationship of influenza virus hemagglutinins in the study. The phylogenetic tree illustrates the relationship among influenza virus HAs that are pertinent to this study. B/Victoria/2/1987 was included as a reference for the influenza B virus HAs. Amino acid sequences were aligned by ClustalW, and the tree was constructed using the neighbor-joining method with Mega 5.10. Bootstrap values for 1,000 replicates are listed at the branches, and the units are the number of amino acid substitutions per site.

FIG. 5A-J shows 1Z105 and 1V270 induce rapid protective immunity to influenza A virus after a single immunization with rHA. (A) BALB/c mice (n=5/group) were immunized i.m. with rPR/8 HA (5 μg/mouse) with the indicated adjuvant or vehicle on day 0. Sera were collected at 7, 14, and 21 days after immunization, as indicated by the red drops. (B to D) At 7, 14, and 21 days post-immunization, HA-specific total IgG (B), IgG1 (C), and IgG2a (D) serum antibody titers were assayed by ELISA with PR/8 virus as a substrate. (E) The Th1-Th2 immune balance shown by the IgG2a/IgG1 ratio expressed on a log 2 scale. (F and G) Three weeks after immunization with rPR/8 HA, the immunized mice (n=10) were administered 10 mLD₅₀ of PR/8 virus and monitored for morbidity, as measured by body weight loss (F), and mortality (G) induced by the viral challenge. (H to J) BALB/c mice (n=5/group) were immunized with rPR/8HA with the indicated adjuvant or vehicle on day 0. (H) The rHA was administered at 5, 1, or 0.2 μg/animal, and serum IgG antibody titers were assessed by ELISA 3 weeks after the immunization. The bars indicate that there was no significant (ns) difference in endpoint titers between mice receiving different antigen doses with the same adjuvant. (I and J) Three weeks after immunization with 0.2 μg/animal of rHA, the immunized mice (n=5) were administered 10 mLD₅₀ of PR/8 virus and monitored for morbidity, as measured by body weight loss (I), and mortality (J) induced by the viral challenge. “No adjuvant” indicates animals that received the antigen in the absence of adjuvant, adjuvant-only animals received both 1Z105 and 1V270 in the absence of antigen, and mice in the vehicle group received an injection of 10% DMSO in PBS without antigen or adjuvant. The data shown are means and SEM; *, P<0.05 compared to no adjuvant by the Kruskal-Wallis test for serum antibody titers. Weight loss data were compared to no adjuvant with multiple t tests, and survival data were compared using a Mantel-Cox test; a P value of 0.05 was considered significant.

FIG. 6A-L shows 1Z105 and 1V270 induce rapid protective immunity to the pH1N1 virus and an avian H5N1 subtype virus and enhance the immunogenicity of Fluzone. (A) BALB/c mice (n=3/group) were immunized with rHA (5 μg/animal) derived from the pH1N1 virus (A/California/04/2009) on day 0 and bled 3 weeks later (red drop). (B) Total serum IgG titers were assayed by ELISA 3 weeks after immunization. (C and D) Three weeks after immunization, the mice were administered 5mLD₅₀ of a mouse-adapted pH1N1 virus (A/Netherlands/602/2009), which is essentially homologous to Cal/09, and monitored for morbidity (C) and mortality (D) induced by the challenge. (E) BALB/c mice (n=10/group) were immunized with rHA derived from a highly pathogenic avian H5N1 virus (A/Vietnam/1203/2004) (2 μg/animal) on day 0. (F) Total serum IgG titers were assayed by ELISA 3 weeks after immunization. (G and H) Three weeks after a single immunization, the mice were challenged with 5 mLD₅₀ of a 6-plus-2 reassortant of the VN/04HAand NA (H5N1) in the PR/8 background and monitored for morbidity (G) and mortality (H) induced by the challenge. The data for the H5N1 challenge were combined from two experiments for a total of 10 mice/group. (I) BALB/c mice (n=10/group) were immunized with 50 ng/HA of 2009-2010 Fluzone. (J to L) Serum IgG levels 3 weeks after immunization to each of the homologous viral components of the vaccine, including A/Brisbane/59/2007 (H1N1) (J), A/Uruguay/716/2007 (H3N2) (K), and B/Brisbane/60/2008 (Victoria lineage) (L), were assayed by ELISA. “No adjuvant” indicates animals that received the antigen in the absence of adjuvant, and mice in the vehicle group received an injection of 10% DMSO in PBS without antigen or adjuvant. The data shown are means and SEM. Weight loss data were compared to no adjuvant with multiple t tests, and survival data were compared using a Mantel-Cox test; P<0.05, which was considered significant.

FIG. 7A-H shows 1Z105 and 1V270 induce sustained protective immunity to influenza A virus after a single immunization with rHA antigen. (A) BALB/c mice were i.m. immunized with rPR/8 HA (5 μg/animal) on day 0 with the indicated adjuvant or vehicle, and sera were collected every 3 weeks (red drops). (B) Levels of antigen-specific total IgG were measured by ELISA. (C) The serum HAI titer to PR/8 virus was assayed at 6 weeks after immunization, with a detection limit of 1:40 serum dilution; nd, not detectable. (D and E) Eighteen weeks after immunization, mice (n=5/group) were administered 10 mLD₅₀ of PR/8 virus and monitored for morbidity, as measured by body weight loss (D), and mortality (E) induced by the viral challenge. (F) (Left) Six months after immunization, all groups of mice were bled (n=10/group), and HAI titers to PR/8 virus were assayed. (F and G) Subsequently, all groups of mice (including adjuvant-only and vehicle groups; n=3/group) were i.m. administered 5 μg/animal (in PBS only) of rPR/8 HA to stimulate a memory response. Five days after exposure to the unadjuvanted protein, the mice were bled to assess HAI to PR/8 virus, with the fold increase over preboost HAI titers indicated in the bars (F, right), and splenocytes were isolated and assayed for ex vivo PR/8 HA-specific antibody production by ELISpot with rPR/8 HA with a different trimerization domain and purification tag from the immunogen as a substrate (G). (H) Six months after immunization in a separate cohort of mice not receiving a protein boost, splenocytes were isolated, stimulated ex vivo overnight with a peptide pool derived from PR/8 HA, and assayed by ELISpot for IFN-γ production. The data shown are means and SEM; *, P<0.05 compared to no adjuvant by Kruskal-Wallis test or nonparametric multiple-contrast test for serum antibody titers and ELISpot assays. Weight loss data were compared to no adjuvant with multiple t tests, and survival data were compared using a Mantel-Cox test; P values of <0.05 were considered significant. *, P<0.05 by t test for fold increase in postboost over preboost HAI titers.

FIG. 8A-I shows 1V270, alone or in combination with 1Z105, induces cross-protective immunity to heterologous influenza viruses. (A) BALB/c mice received a single immunization of rPR/8 HA (5 μg/animal) on day 0. (B) Three weeks after immunization, the cross-reactive serum IgG was assayed by ELISA using as a substrate rCal/09 HA, which has a trimerization domain and purification tag different than those utilized for immunization. (C) Total serum IgG titers reactive to pandemic H1 and PR/8 HAs, both purified rHAs with a trimerization domain (GCN4 leucine zipper) and a purification tag (streptavidin purification domain) different than those of the PR/8 immunogen (T4 phage fibritin natural trimerization domain and C-terminal 6× His tag), were determined by ELISA and presented as the PR/8-to-Cal/09 endpoint titer (EPT) ratio. (D) IgG2a and IgG1 serum antibodies cross-reactive to the Cal/09 HA were assayed by ELISA and presented as the IgG2a/gG1 ratio. (E and F) Four weeks after immunization, mice (n=20/group) were administered 5 mLD₅₀ of a heterologous H1N1 virus, a mouse-adapted pandemic H1N1 strain (A/Netherlands/602/2009), and monitored for morbidity, as measured by body weight loss (E), and mortality (F) induced by the viral challenge. (G) BALB/c mice (10 mice/group) were immunized with 2009-2010 Fluzone, containing B/Brisbane/60/2008 (Victoria lineage), with the indicated adjuvant or vehicle. (H and I) Mice were challenged 3 to 4 weeks after immunization with 25 mLD₅₀ of a heterologous mouse-adapted virus, B/Florida/04/2006 (Yamagata lineage), and monitored for morbidity, as measured by body weight loss (H), and mortality (I) induced by the viral challenge. The data were combined from two independent challenges. The data shown are means and SEM; *, P<0.05 compared to no adjuvant by a nonparametric multiple-contrast test for serum antibody titers. PR/8-to-Cal/09 endpoint titer ratios were not significantly (ns) different by one-way ANOVA. Weight loss data were compared to no adjuvant with multiple t tests, and survival data were compared using a Mantel-Cox test; a P value of <0.05 was considered significant.

FIG. 9A-H shows 1V270, alone or in combination with 1Z105, induces protective heterosubtypic immunity based upon the conserved HA stalk domain. (A to C) (A) Vaccination scheme for the induction of broadly protective and HA stalk-specific immunity using chimeric hemagglutinins. Three sequential immunizations with rHA (5 μg/animal), with each stalk component derived from A/Puerto Rico/8/1934, included a prime with cH6/1PR/8 followed by boosts with H1 (PR/8 strain) and cH2/1PR/8 proteins administered with the indicated adjuvant or vehicle control. The immunized animals remained naive to the subtype H5 globular head in the heterosubtypic challenge virus, a 6-plus-2 reassortant of A/Vietnam/1203/2004 (H5N1) in the PR/8 background, in order to assay for reactivity and protection on the basis of the conserved group I HA stalk domain. Three weeks after the third immunization, sera were collected (A), drop above line), and total IgG serum titers to the H5 subtype HA (individual mouse sera) (B) and the cH5/3 HA (pooled group sera) (C) were assayed by ELISA. (D) IgG1 and IgG2a serum titers (presented as the IgG2a/IgG1 ratio) cross-reactive to the heterosubtypic H5 subtype HA (VN/04) were assayed by ELISA. (E and F) Subsequently, the mice (n=7/group) were administered 10 mLD₅₀ of the reassortant H5N1 virus and monitored for morbidity, as measured by body weight loss (E), and mortality (F) induced by the viral challenge. (G and H) The sera were further analyzed by ELISA for reactivity to more divergent group I HAs, including subtype H11 (G) and H12 (H) viruses. “No adjuvant” indicates animals that received the antigen in the absence of adjuvant, adjuvant-only animals received both 1Z105 and 1V270 in the absence of antigen, and mice in the vehicle group received an injection of 10% DMSO in PBS without antigen or adjuvant. The data shown are means and SEM; *, P<0.05 compared to no adjuvant by a Kruskal-Wallis test for serum antibody titers. Weight loss data were compared to no adjuvant with multiple t tests, and survival data were compared using a Mantel-Cox test; P values of <0.05 were considered significant.

FIG. 10A-C shows a combination of 1Z105 and 1V270 induces less local and systemic inflammatory response than AddaVax. BALB/c mice (n=3 or 4) were injected in the gastrocnemius muscles with 1Z105 (89.4 μg/dose) and 1V270 (10.8 μg/dose), 1V270 (10.8 μg/dose), 1Z105 (89.4 μg/dose), AddaVax (1:1 with saline), or vehicle (10% DMSO in saline) in a 50-μl volume. Twenty-four hours after injection, muscles and sera were harvested. (A) The H&E-stained sections of the injected muscles were examined for cell infiltration. Shown are the muscles injected with 1Z105 plus 1V270 (original magnification, ×200) (i), 1Z105 plus 1V270 (×400) (ii), 1V270 (×200) (iii), 1Z105 (×200) (iv), AddaVax (×200) (v), AddaVax (×400) (vi), and vehicle (×200) (vii). Scale bars, 100 μm. (ii and vi) The white arrows indicate mononuclear cells, and the black arrows indicate polymorphonuclear cells. (B) Gene expression of cytokines and chemokines at the site of injection was determined 24 h after injection. RNA was isolated from muscles, and mRNAs specific for IL-6, KC, MCP-1, and MIP-1α were quantitated by RT-PCR. (C) Systemic cytokine levels (IL-6 and KC) 24 h after injection were examined by Luminex bead assay. *, P<0.05 compared to the vehicle-injected group by one-way ANOVA. The error bars indicate SEM.

### DETAILED DESCRIPTION

As used herein and in the appended claims, the singular forms “a,” “and,” and “the” include plural referents unless the context clearly dictates otherwise. Thus, for example, reference to “an antigen” includes a plurality of such antigen and reference to “the adjuvant” includes reference to one or more adjuvants, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Many antigens are poorly immunogenic and require the use of adjuvants to promote a strong immune response. Numerous adjuvants are known and used, however, improved adjuvants are desirable. The disclosure provides compositions that promote an immune response when administered in combination with an antigen. The disclosure exemplifies the adjuvants in combination with antigens from influenza.

One of the most effective ways to protect against influenza virus infection is through vaccination. However, since influenza viruses are constantly undergoing change (antigenic drift), it is difficult to predict what subtype and strain of virus will be circulating in the next influenza season or in the next pandemic, and to allow sufficient time (about 6 months) for vaccine manufacture and distribution of conventional vaccines. Thus, the effectiveness of a conventional vaccine against seasonal influenza may be limited to the subtype and strain that was correctly predicted at the time of vaccine manufacture, well before the beginning of the influenza season. These conventional vaccines are typically based on antigens associated with the influenza hemagglutinin (HA) protein, and in particular, the globular head domain of the protein. This highly antigenic head domain is variable across strains and subtypes of influenza viruses and thus, an immune response against one globular head domain subtype might be limited to that particular head domain and fail to provide an adequate immune response against a virus strain having a different head domain. Influenza HA antigens derived from the stem or stalk domain of the protein, which are more highly conserved across virus strains, are generally less immunogenic than the head domain antigens that are typically dominant in the conventional vaccines and therefore there is a need to augment the immunogenicity of these HA stalk antigens to a level that would generate an adequate immune response in the host, resulting in a response against multiple influenza strains.

The disclosure provides compositions and methods useful for improving an immune response. The composition comprises two Toll-Like Receptor (TLR) agonists in combination with an antigen. The two TLR agonists can be different TLR agonists (e.g., agonists to different Toll-like Receptors) or can be two different TLR agonists directed against the same Toll-Like Receptor. The disclosure exemplifies the adjuvant compositions of the disclosure using influenza antigens. Thus, in one embodiment, the disclosure provides compositions and methods useful for improving an immune response against the more conserved “HA stalk antigens”. The disclosure demonstrates that combining (i) the HA stalk antigens, such as those described in International Application No. WO 2010/117786, the disclosure of which is incorporated by reference herein, with (ii) a TLR4 small molecule adjuvant (disclosed in WO 2014/052828, the disclosure of which is incorporated herein) and (iii) with a TLR7 small molecule adjuvant (disclosed in WO 2011/139348, the disclosure of which is incorporated by reference herein) resulted in both homologous and heterologous protection against influenza virus infections in animal models and this protection was more effective in comparison to a conventional adjuvant such as AddaVax. The combined use of a HA stalk antigen with synthetic small molecule adjuvants described herein in a vaccine generates a rapid, broad spectrum response that provides superior protection against influenza infections. The compositions, methods and data provided herein demonstrate that the combination of TLR small molecule agonists with an antigen can provide an effective and strong immune response.

“TLR” generally refers to any Toll-like receptor of any species of organism. These include TLR1, TLR2, TLR3, TLR4, TLR5, TLR6, TLR7, TLR8, TLR9, TLR10 and TLR11. A specific TLR may be identified with additional reference to species of origin (e.g., human, murine, etc.), a particular receptor (e.g., TLR6, TLR7, TLR8, etc.), or both. TLRs are components of the innate immune system that regulate NF_{κ}B activation. Thus, compounds comprising TLR agonists or antagonists can be identified using well recognized NF_{κ}B assays. For example, assays for detecting TLR agonism of test compounds are described, for example, in U.S. Provisional Patent Application Ser. No. 60/432,650, filed Dec. 11, 2002, and recombinant cell lines suitable for use in such assays are described, for example, in U.S. Provisional Patent Application Ser. No. 60/432,651, filed Dec. 11, 2002 incorporated by reference herein.

Various TLRs are known. For example, “TLR2” as used herein refers to the product (NCBI Accession AAH33756.1) of the TLR2 gene, and homologs and functional fragments thereof. “TLR3” as used herein refers to the product (NCBI Accession ABC86910.1) of the TLR3 gene, and homologs and functional fragments thereof. “TLR4” as used herein refers to the product of the TLR4 gene, and homologs, isoforms, and functional fragments thereof: Isoform 1 (NCBI Accession NP_{—}612564.1); Isoform 2 (NCBI Accession NP_{—}003257.1); Isoform 3 (NCBI Accession NP_{—}612567.1). “TLR5” as used herein refers to the product (NCBI Accession AAI09119) of the TLR5 gene, and homologs, and functional fragments thereof. “TLR7” as used herein refers to the product (NCBI Accession AAZ99026) of the TLR7 gene, and homologs, and functional fragments thereof. “TLR8” as used herein refers to the product (NCBI Accession AAZ95441) of the TLR8 gene, and homologs, and functional fragments thereof. “TLR9” as used herein refers to the product (NCBI Accession AAZ95520) of the TLR9 gene, and homologs, and functional fragments thereof.

TLR4 recognizes lipopolysaccharide (LPS) from Gram-negative bacteria. The recognition process is enhanced by LPS-binding protein (LBP), which carries LPS to the CD 14 molecule, where it is then presented to the MD-2-TLR4 complex. See e.g., Latz, E., et al., J. Biol. Chem. 2002, 277:47834-47843. TLR4 is expressed predominately on monocytes, mature macrophages and dendritic cells, mast cells and the intestinal epithelium. TLR modulators (antagonists) for TLR4 include NI-0101 (Hennessy 2010, Id.), 1A6 (Ungaro, R., et al., Am. J. Physiol. Gastrointest. Liver Physiol. 2009, 296:G1167-G1179), AV411 (Ledeboer, A., et al., Neuron Glia Biol. 2006, 2:279-291; Ledeboer, A., et al., Expert Opin. Investig. Drugs 2007, 16:935-950), Eritoran (Mullarkey, M., et al., J. Pharmacol. Exp. Ther. 2003, 305:1093-1102), and TAK-242 (Li, M., et al., Mol. Pharmacol. 2006, 69:1288-1295). TLR modulators (agonists) for TLR4 include Pollinex® Quattro (Baldrick, P., et al., J. Appl. Toxicol. 2007, 27:399-409; DuBuske, L., et al., J. Allergy Clin. Immunol. 2009, 123:S216).

TLR7 and TLR8 are found in endosomes of monocytes and macrophages, with TLR7 also being expressed on plasmacytoid dendritic cells, and TLR8 also being expressed in mast cells. Both these receptors recognize single stranded RNA from viruses. Synthetic ligands, such as R-848 and imiquimod, can be used to activate the TLR7 and TLR8 signaling pathways. See e.g., Caron, G., et al., J. Immunol. 2005, 175:1551-1557. TLR9 is expressed in endosomes of monocytes, macrophages and plasmacytoid dendritic cells, and acts as a receptor for unmethylated CpG islands found in bacterial and viral DNA. Synthetic oligonucleotides that contain unmethylated CpG motifs are used to activate TLR9. For example, class A oligonucleotides target plasmacytoid dendritic cells and strongly induce IFNa production and antigen presenting cell maturation, while indirectly activating natural killer cells. Class B oligonucleotides target B cells and natural killer cells and induce little interferon-a (IFNa). Class C oligonucleotides target plasmacytoid dendritic cells and are potent inducers of IFNa. This class of oligonucleotides is involved in the activation and maturation of antigen presenting cells, indirectly activates natural killer cells and directly stimulates B cells. See e.g., Vollmer, J., et al., Eur. J. Immunol. 2004, 34:251-262; Strandskog, G., et al., Dev. Comp. Immunol. 2007, 31:39-51. 20 Reported TLR modulators (agonist) for TLR7 include ANA772 (Kronenberg, B. & Zeuzem, S., Ann. Hepatol. 2009, 8:103-112), Imiquimod (Somani, N. & Rivers, J. K., Skin Therapy Lett. 2005, 10:1-6), and AZD8848 (Hennessey 2010, Id.) TLR modulators (agonist) for TLR8 include VTX-1463 (Hennessey 2010, Id.) TLR modulators (agonist) for TLR7 and TLR8 include Resiquimod (Mark, K. E., et al., J. Infect. Dis. 2007, 195:1324-1331; Pockros, P. J., et al., J. Hepatol. 2007, 47:174-182). TLR modulators (antagonists) for TLR7 and TLR9 include IRS-954 (Barrat, F. J., et al., Eur. J. Immunol. 2007, 37:3582-3586), and IMO-3100 (Jiang, W., et al., J. Immunol. 2009, 182:48.25). TLR9 agonists include SD-101 (Barry, M. & Cooper, C., Expert Opin. Biol. Ther. 2007, 7:1731-1737), IMO-2125 (Agrawal, S. & Kandimalla, E. R., Biochem. Soc. Trans. 2007, 35:1461-1467), Bio Thrax plus CpG-7909 (Gu, M., et al., Vaccine 2007, 25:526-534), AVE0675 (Parkinson, T., Curr. Opin. Mol. Ther. 2008, 10:21-31), QAX-935 (Panter, G., et al., Curr. Opin. Mol Ther. 2009, 11:133-145), SAR-21609 (Parkinson 2008, Id.), and DIMS0150 (Pastorelli, L., et al., Expert Opin. Emerg. Drugs 2009, 14:505-521).

The terms “TLR modulator,” “TLR immunomodulator” and the like as used herein refer to compounds which agonize or antagonize a Toll-Like Receptor (See e.g., PCT/US2010/000369; Hennessy, E. J., et al., Nature Reviews, 9:283-307, 2010; PCT/US2008/001631; PCT/US2006/032371; and PCT/US2011/000757). Accordingly, a “TLR agonist” is a TLR modulator which agonizes a TLR, and a “TLR antagonist” is a TLR modulator which antagonizes a TLR.

A “TLR agonist” refers to a compound that acts as an agonist of a TLR. This includes TLR1, TLR2, TLR3, TLR4, TLR5, TLR6, TLR7, TLR8, TLR9, TLR10, and TLR11 agonists or a combination thereof. A TLR agonist can include non-naturally occurring molecules that agonize a Toll-Like Receptor. The agonistic activity need not be equal to a naturally occurring ligand of the TLR so long as it has at least some agonistic activity. Unless otherwise indicated, reference to a TLR agonist compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers. Also, a compound may be identified as an agonist of one or more particular TLRs (e.g., a TLR7 agonist, a TLR8 agonist, or a TLR7/8 agonist). In addition, a composition can include one or more TLR agonists. For example, a composition can comprise a TLR4 agonist and a TLR7 agonist. For example, in one embodiment, a composition can comprise an emulsion containing at least two different TLR agonists and a water soluble antigen.

In one embodiment, the TLR agonist comprises a TLR7 agonist having the general structure of Formula I:

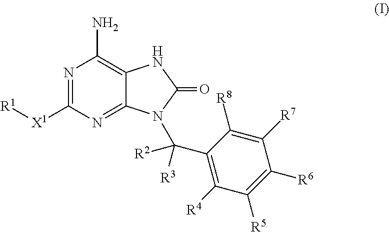

wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉)heterocyclic, substituted (C₅₋₉) heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O) OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or

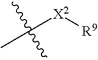

and wherein at least one of R⁴-R⁸ is

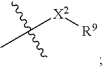

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof.

In one embodiment, R⁹ can comprise a group having the general structure of a phospholipid:

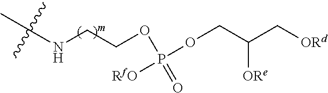

wherein R^{d} and R^{e} are each independently a hydrogen or an acyl group, R^{f} is a negative charge or a hydrogen, and m is 1 to 8, wherein a wavy line indicates a position of bonding, wherein an absolute configuration at the carbon atom bearing OR^{e} is R, S, or any mixture thereof. For example, m can be 1, providing a glycerophosphatidylethanolamine. More specifically, R^{d} and R^{e} can each be oleoyl groups. A “phospholipid” as the term is used herein refers to a glycerol mono- or diester bearing a phosphate group bonded to a glycerol hydroxyl group with an alkanolamine group being bonded as an ester to the phosphate group, of the general formula.

In various embodiments, a phospholipid of R⁹ can comprise two carboxylic esters and each carboxylic ester includes one, two, three or four sites of unsaturation, epoxidation, hydroxylation, or a combination thereof.

In various embodiments, the phospholipid of R⁹ can comprise two carboxylic esters and the carboxylic esters of are the same or different. More specifically, each carboxylic ester of the phospholipid can be a C₁₇ carboxylic ester with a site of unsaturation at C₈-C₉. Alternatively, each carboxylic ester of the phospholipid can be a C₁₈ carboxylic ester with a site of unsaturation at C₉-C₁₀.

In various embodiments, R⁹ can be dioleoylphosphatidyl ethanolamine (DOPE).

In various embodiments, R⁹ can be 1,2-dioleoyl-sn-glycero-3-phospho ethanolamine and X² can be C(O).

In various embodiments, X² can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

In various embodiments, X² can be C(O), or can be selected from the group consisting of:

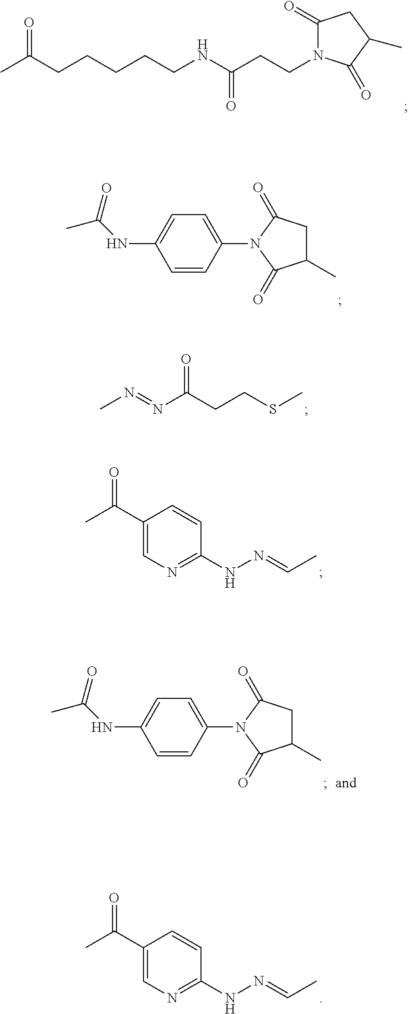

In various embodiments, the compound of Formula (I) can be defined such that it provides formula I(a):

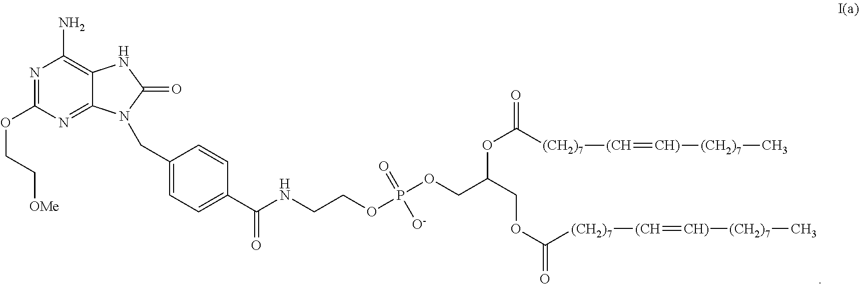

In various embodiments, the compound of formula I(a) can be the R-enantiomer of the above structure:

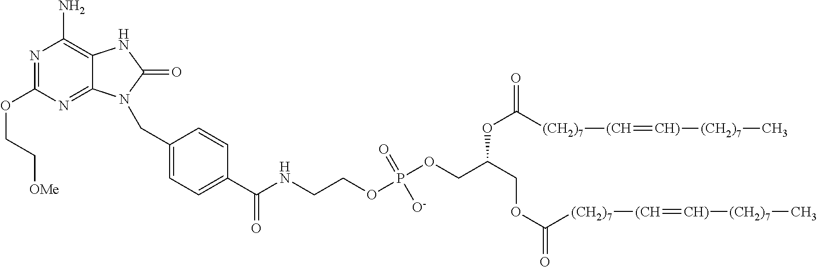

Within the disclosure it is to be understood that a compound of the formulas or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

In one embodiment, the TLR agonist is a TLR4 agonist having the general structure of Formula II:

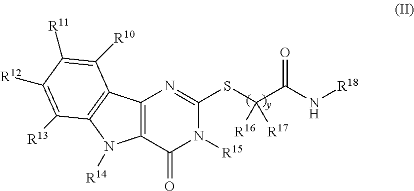

or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5.

In one embodiment, R¹⁵ is R^{15A}-(substituted or unsubstituted cycloalkyl), R^{15A}-(substituted or unsubstituted heterocycloalkyl), R^{15A}-(substituted or unsubstituted aryl), or R^{15A}-(substituted or unsubstituted heteroaryl), wherein R^{15A} is independently halogen, —CN, —CF₃, —CCl₃, —OH, —NH₂, —SO₂, —COOH, oxo, nitro, —SH, —CONH₂, R^{15B}-(substituted or unsubstituted alkyl), R^{15B}-(substituted or unsubstituted heteroalkyl), R^{15B}-(substituted or unsubstituted cycloalkyl), R^{15B}-(substituted or unsubstituted heterocycloalkyl), R^{15B}-(substituted or unsubstituted aryl), or R^{15B}-(substituted or unsubstituted heteroaryl), wherein R^{15B} is independently halogen, —CN, —CF₃, —CCl₃, —OH, —NH₂, —SO₂, —COOH, oxo, nitro, —SH, —CONH₂, R^{15C}-(substituted or unsubstituted alkyl), R^{15C}-(substituted or unsubstituted heteroalkyl), R^{15C}-(substituted or unsubstituted cycloalkyl), R^{15C}-(substituted or unsubstituted heterocycloalkyl), R^{15C}-(substituted or unsubstituted aryl), or R^{15C}-(substituted or unsubstituted heteroaryl), wherein R^{15C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R¹⁸ is R^{18A}-(substituted or unsubstituted cycloalkyl), R^{18A}-(substituted or unsubstituted heterocycloalkyl), R^{18A}-(substituted or unsubstituted aryl), or R^{18A}-(substituted or unsubstituted heteroaryl), wherein R^{18A} is independently halogen, —CN, —CF₃, —CCl₃, —OH, —NH₂, —SO₂, —COOH, oxo, nitro, —SH, —CONH₂, R^{15B}-(substituted or unsubstituted alkyl), R^{15B}-(substituted or unsubstituted heteroalkyl), R^{18B}-(substituted or unsubstituted cycloalkyl), R^{18B}-(substituted or unsubstituted heterocycloalkyl), R^{18B}-(substituted or unsubstituted aryl), or R^{18B}-(substituted or unsubstituted heteroaryl), wherein R^{18B} is independently halogen, —CN, —CF₃, —CCl₃, —OH, —NH₂, —SO₂, —COOH, oxo, nitro, —SH, —CONH₂, R^{18C}-(substituted or unsubstituted alkyl), R^{18C}-(substituted or unsubstituted heteroalkyl), R^{18C}-(substituted or unsubstituted cycloalkyl), R^{18C}-(substituted or unsubstituted heterocycloalkyl), R^{18C}-(substituted or unsubstituted aryl), or R^{18C}-(substituted or unsubstituted heteroaryl), wherein R^{18C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R¹⁴ is hydrogen, or R^{14A}-(substituted or unsubstituted alkyl). R^{14A} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, each of R¹⁰-R¹³ is independently halogen, —CN, —SH, —OH, —COOH, —NH₂, —CONH₂, nitro, —CF₃, —CCl₃, R^{10-13A}-(substituted or unsubstituted alkyl), R^{4A}-substituted or unsubstituted heteroalkyl, R^{10A-13A}-(substituted or unsubstituted cycloalkyl), R^{10A-13A}-(substituted or unsubstituted heterocycloalkyl), R^{10-13A}-(substituted or unsubstituted aryl), or R^{10-13A}-(substituted or unsubstituted heteroaryl). R^{10-13A} is independently halogen, —CN, —CF₃, —CCl₃, —OH, —NH₂, —SO₂, —COOH, oxo, nitro, —SH, —CONH₂, R^{10-13B}-(substituted or unsubstituted alkyl), R^{10-13B}-(substituted or unsubstituted heteroalkyl), R^{10-13B}-(substituted or unsubstituted cycloalkyl), R^{10-13B}-(substituted or unsubstituted heterocycloalkyl), R^{10-13B}-(substituted or unsubstituted aryl), or R^{10-13B}-(substituted or unsubstituted heteroaryl). R^{10-13B} is independently halogen, —CN, —CF₃, —CCl₃, —OH, —NH₂, —SO₂, —COOH, oxo, nitro, —SH, —CONH₂, R^{10-13C}-(substituted or unsubstituted alkyl), R^{10-13C}-(substituted or unsubstituted heteroalkyl), R^{10-13C}-(substituted or unsubstituted cycloalkyl), R^{10-13C}-(substituted or unsubstituted heterocycloalkyl), R^{10-13C}-(substituted or unsubstituted aryl), or R^{10-13C}-(substituted or unsubstituted heteroaryl). R^{10-13C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to any embodiment disclosed above, in one embodiment, R¹⁵ is not a substituted phenyl. In another embodiment, R¹⁵ is not a p-fluorophenyl or p-methylphenyl.

Further to any embodiment disclosed above, in one embodiment, R¹⁸ is not a substituted or unsubstituted aryl, unsubstituted cyclohexyl, unsubstituted thiazole, or —CH₂-furanyl.

In a specific embodiment, the TLR4 agonist has the structure of Formula II(a):

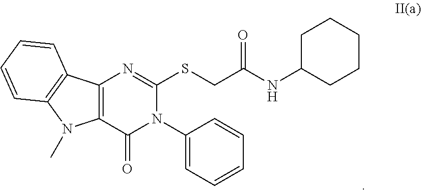 [SMILES: Cn1c2ccccc2c2nc(SCC(=O)NC3CCCCC3)n(-c3ccccc3)c(=O)c21]

In another embodiment there is provided a compound with structure of Formula (III):

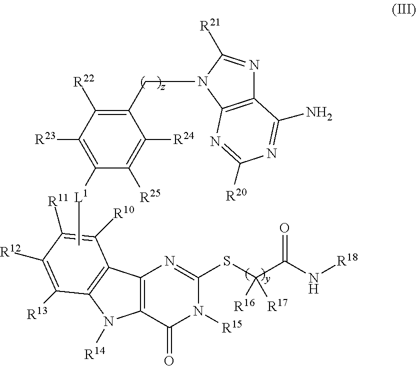

For Formula (III), R¹⁰⁻¹³, R¹⁴, R¹⁵, R¹⁸, and y are as disclosed above for Formula (II), including embodiments thereof, with the proviso that one of R¹⁰⁻¹³ forms a bond with L1; and wherein z is an integer from 1 to 10.

In one embodiment, z is an integer from 1 to 3. In one embodiment, z is 1.

In one embodiment, L¹ is a substituted or unsubstituted alkylene, or a substituted or unsubstituted heteroalkylene. In one embodiment, L¹ includes a water soluble polymer. A “water soluble polymer” refers to a polymer which is sufficiently soluble in water under physiologic conditions of e.g., temperature, ionic concentration and the like, as known in the art, to be useful for the methods described herein. An exemplary water soluble polymer is polyethylene glycol. In one embodiment, the water soluble polymer is —(OCH₂CH₂)ₘ— wherein m is 1 to 100. In one embodiment, L¹ includes a cleavage element. A “cleavage element” is a chemical functionality which can undergo cleavage (e.g., hydrolysis or by enzymatically cleavable). The terms “enzymatically cleavable” and the like refer, in the usual and customary sense, to a chemical moiety which can undergo bond scission by the action of an enzyme, e.g., hydrolase, esterase, lipase, peptidase, amidase and the like. Scission can occur at a terminal bond of L¹ or a non-terminal bond within L¹. Bond scission of L¹ can be accompanied by bond rearrangement of the resulting fragments of L¹ and bond addition, e.g., addition of water (e.g., under the action of a hydrolase, esterase, lipase, peptidase, amidase and the like). Enzymatic cleavage can occur under physiological conditions, e.g., under the action of a physiological enzyme within an organism. Enzymatic cleavage can occur within a cell, e.g., a biological cell as disclosed herein. Enzymatic cleavage can occur extracellularly, e.g., in the circulatory system 20 of a subject. Enzymatic cleavage can occur under in vitro conditions.

In one embodiment, L¹ is —C(O)—X¹-L^{1A}-X²—C(O)—, wherein X¹ and X² are —O— or —NH—, and L^{1A} is substituted or unsubstituted alkylene or substituted or unsubstituted heteroalkylene. In one embodiment, L^{1A} is -L^{1B}-(CH₂CH₂O)ₙ— wherein n is an integer from 1 to 100, and L^{1B} is unsubstituted C₁-C₁₀ alkylene. In one embodiment, n is an integer in the range of about 1 to 100, 1 to 90, 1 to 80, 1 to 70, 1 to 60, 1 to 50, 1 to 40, 1 to 30, 1 to 20, or 1 to 10. In one embodiment, n is about 100, 90, 80, 70, 60, 50, 40, 30, 20, 18, 16, 14, 12, 10, or 9, 8, 7, 6, 5, 4, 3, or 2. In one embodiment, n is an integer in the range of about 1 to 100, 1 to 90, 1 to 80, 1 to 70, 1 to 60, 1 to 50, 1 to 40, 1 to 30, 1 to 20, or 1 to 10, and L^{1B} is ethylene. In one embodiment, n is an integer from 1 to 10, and L^{1B} is ethylene. In one embodiment, L¹ is 30 —C(O)O—CH₂CH₂—(OCH₂CH₂)ₙ—NH—C(O)—, wherein n is 1 to 10, e.g., 1, 2, 3, 4, 5, 6, 7, 8, 9, or 10.

In another embodiment, there is provided a compound with structure of Formula (V):

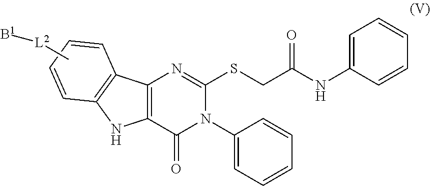

wherein L² is a linker, and B¹ is a purine base or analog thereof.

In one embodiment, L² is a substituted or unsubstituted alkylene, or a substituted or unsubstituted heteroalkylene. In one embodiment, L² includes a water soluble polymer. A “water soluble polymer” refers to a polymer which is sufficiently soluble in water under physiologic conditions of e.g., temperature, ionic concentration and the like, as known in the art, to be useful for the methods described herein. An exemplary water soluble polymer is polyethylene glycol. In one embodiment, the water soluble polymer is —(OCH₂CH₂)ₘ— wherein m is 1 to 100. In one embodiment, L² includes a cleavage element. A “cleavage element” is a chemical functionality which can undergo cleavage (e.g., hydrolysis) to release the compound of Formula (V), optionally including remnants of linker L², and B¹, optionally including remnants 20 of linker L².

A representative schematic synthesis of a compound of Formula (IV(b)) is depicted in Scheme 1, wherein element (I(b)) is a modified versatile intermediate TLR7 agonist, element (II(b)) is a modified TLR7 agonist with linker, and element (IV(b)) is a TLR4-TLR7 dual ligand conjugate (cleavable linkage shown). Methods of conjugation of elements (I(b)) and (II(b)) are well known in the art to afford the resulting dual ligand conjugate with the structure of Formula (IVb). The R groups are as defined above, with the proviso that one of R10-R13 is a COOH group and that this COOH group forms an ester bond with the terminal hydroxyl group of Formula I(b).

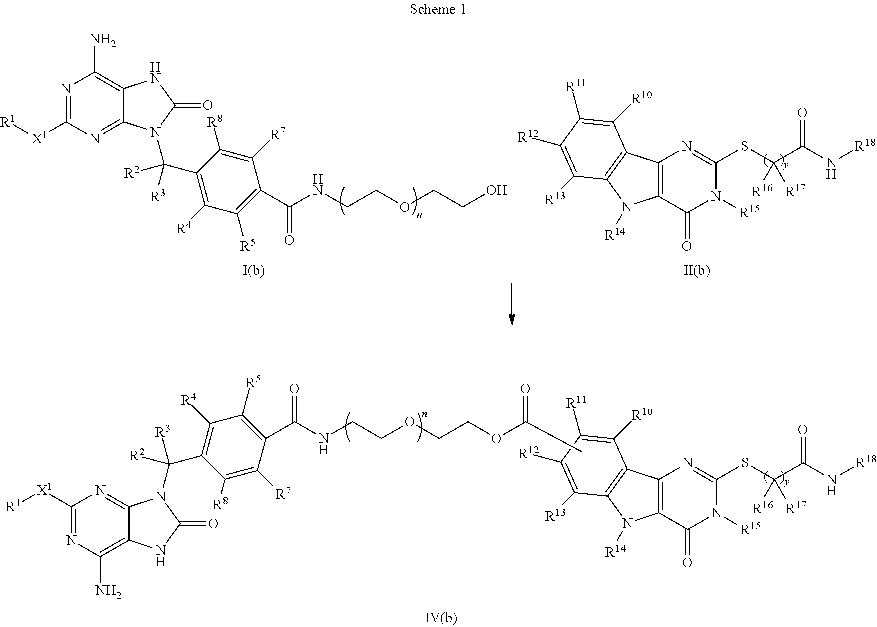

In one embodiment, the disclosure provides a prophylactic or therapeutic method for preventing or treating a pathological condition or symptom in a mammal, such as a human, with one or more antigens and one or more adjuvants. The method includes administering to a mammal in need of such therapy, an antigen and an effective amount of one or more adjuvants at least one adjuvant composition comprising a plurality of TLR agonists (e.g., TLR-4 and TLR-7 agonists), or a pharmaceutically acceptable salt thereof. Non-limiting examples of pathological conditions or symptoms that are suitable for treatment include cancers, microbial infections or diseases, e.g., skin or bladder diseases. In one embodiment, the adjuvants of the disclosure can be used to prepare vaccines against bacteria, viruses, cancer cells, or cancer-specific peptides, as a CNS stimulant, or for biodefense. The disclosure thus provides an adjuvant for use alone or with other therapeutic agents in medical therapy (e.g., for use as an anti-cancer agent, to prevent, inhibit or treat bacterial diseases, to prevent, inhibit or treat viral diseases, such as hepatitis C and hepatitis B, and generally as agents for enhancing the immune response).

The disclosure demonstrates that a combination of TLR agonists is effective and unexpectedly beneficial as adjuvants. In one embodiment, the combination of adjuvants comprises a TLR7 agonist as set forth in Formula I (or salt thereof) and a TLR4 agonist as set forth in Formula II (or salt thereof). The two agonist can be covalently linked as in Formula IV(b), but need not be linked and can be formulated together without a covalent linkage.

In one embodiment, the disclosure provides a method for preventing, inhibiting or treating cancer by administering an effective amount of a cancer antigen and a TLR4 agonist and a TLR7 agonist. The cancer may be an interferon sensitive cancer, such as, for example, a leukemia, a lymphoma, a myeloma, a melanoma, or a renal cancer. Specific cancers that can be treated include melanoma, superficial bladder cancer, actinic keratoses, intraepithelial neoplasia, and basal cell skin carcinoma, squamous, and the like. In addition, the method of the disclosure includes treatment for a precancerous condition such as, for example, actinic keratoses or intraepithelial neoplasia, familial polyposis (polyps), cervical dysplasia, cervical cancers, superficial bladder cancer, and any other cancers associated with infection (e.g., lymphoma Karposi's sarcoma, or leukemia); and the like.

In another embodiment, the disclosure provides a method to prevent or inhibit a gram-positive bacterial infection in a mammal, comprising administering to the mammal an effective amount of a composition comprising a bacterial antigen of a gram-positive bacteria and an amount of a TLR4 agonist, and a TLR7 agonist. In one embodiment, a TLR4 agonist and a TLR7 agonist is administered with one or more antigens.

Other disorders that may be amenable to treatment with an antigen and a plurality of TLR agonists include, but are not limited to Multiple Sclerosis, lupus, rheumatoid arthritis, Crohn's Disease and the like.

In one embodiment, the two agonists are prepared in a biologically compatible hydrophobic/non-polar buffer (e.g., a lipid or oil buffer). The hydrophobic buffer can be used to generate a nanoemulsion upon mixture with an aqueous buffer (e.g., a buffer comprising an antigen/immunogen). Thus, in one embodiment, the disclosure provides a formulation comprising a combination of a TLR7 agonist, as set forth in Formula I, and a TLR4 agonist, as set forth in Formula II, in a hydrophobic/non-polar biologically compatible buffer. As described more fully herein, an aqueous/polar buffer comprising an antigen/immunogen to which an immune response is desired is mixed with the TLR-agonist-formulation to form a nanoemulsion vaccine that can then be delivered/administered to a subject to be vaccinated.

The term “suspension,” as used herein refers to a mixture of two materials the generally separate over time. The time period of separation may be 5 seconds to several hours or days and will depend upon the temperature, size and weight of the various components and the like. A suspension can be comprised of two immiscible solvent or can be a solvent and a particulate. In a suspension one component “sediments” out over time.

The term “nanoemulsion,” as used herein, includes dispersions or droplets, as well as other lipid structures that can form as a result of hydrophobic forces that drive polar residues (i.e., long hydrocarbon chains) away from water and drive polar head groups toward water, when a water immiscible oily phase is mixed with an aqueous phase. An emulsion refers to a substantially even dispersion of non-miscible components. These other lipid structures include, but are not limited to, unilamellar, paucilamellar, and multilamellar lipid vesicles, micelles, and lamellar phases. In an emulsion the two phases separate and coalesce over time.

The disclosure provides methods, compositions and kits for the stimulation of an immune response to an antigen/immunogen. The disclosure demonstrates that nanoemulsion and/or suspension vaccines of the disclosure comprising a TLR4 and TLR7 agonist can surprisingly increase the immune response of a subject to the antigen/immunogen provided therein.

The nanoemulsion and/or nanoemulsion and/or suspension vaccine of the disclosure comprises droplets having an average diameter size, less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm.

The aqueous buffer containing the antigen/immunogen to be used (which upon generating the nanoemulsion comprises the aqueous phase of the emulsion) can comprise any type of aqueous buffer including, but not limited to, water (e.g., distilled water, purified water, water for injection, de-ionized water, tap water etc.) and solutions (e.g., phosphate buffered saline (PBS) solution etc.). In certain embodiments, the aqueous phase comprises water at a pH of about 4 to 10, but typically about 6 to 8. The aqueous phase may further be sterile and pyrogen free.

Organic solvents present in the nanoemulsion vaccines of the disclosure include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one embodiment of the disclosure, the organic solvent is an alcohol chosen from a nonpolar solvent, a polar solvent, a protic solvent, or an aprotic solvent.

Suitable organic solvents for the nanoemulsion and/or suspension vaccine include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, semi-synthetic derivatives thereof, and any combination thereof.

The biocompatible hydrophobic buffer containing the TLR-agonist(s) which forms the “oil” phase in the nanoemulsion and/or suspension vaccine of the disclosure can be any cosmetically or pharmaceutically acceptable oil-based buffer. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, famesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

The surfactant that may be present in the nanoemulsion and/or suspension vaccine of the disclosure can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and typically, between about 10 and about 100. Typically, the alkoxylated alcohol is the species wherein R₅ is a lauryl group and y has an average value of 23.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, other Triton surfactants, Tween-based surfactants, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride, Alkyl dimethyl benzyl ammonium chloride, Alkyl didecyl dimethyl ammonium chloride, Alkyl dimethyl benzyl ammonium chloride, dialkyl dimethyl benzyl ammonium chloride, Alkyl dimethyl ethyl ammonium bromide, Alkyl dimethyl ethylbenzyl ammonium chloride, Alkyl dimethyl isopropylbenzyl ammonium chloride, Alkyl trimethyl ammonium chloride, Alkyldimethyl(ethylbenzyl) ammonium chloride, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl)octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the disclosure are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecyl amine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, CHAPSO, 3-(Decyldimethylammonio)propanesulfonate inner salt, 3-Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio) propanesulfonate, semi-synthetic derivatives thereof, and combinations thereof.

In some embodiments, the nanoemulsion and/or suspension vaccine comprises a cationic surfactant, which can be cetylpyridinium chloride. In other embodiments of the disclosure, the nanoemulsion and/or suspension vaccine comprises a cationic surfactant, and the concentration of the cationic surfactant is less than about 5.0% and greater than about 0.001%. In yet another embodiment of the disclosure, the nanoemulsion and/or suspension vaccine comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion and/or suspension vaccine is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion and/or suspension vaccine is less than about 5.0% and greater than about 0.001%.

In another embodiment of the disclosure, the nanoemulsion and/or suspension vaccine comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the disclosure, the nanoemulsion and/or suspension vaccine comprises a cationic surfactant present in a concentration of about 0.01% to about 2%, in combination with a nonionic surfactant.

Additional compounds suitable for use in the nanoemulsion and/or suspension vaccines of the disclosure include, but are not limited to, one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

Suitable preservatives in the nanoemulsion and/or suspension vaccines of the disclosure include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis(p-chlorophenyldiguanido)hexane), chlorphenesin (3-(-4-chloropheoxy)-propane-1,2-diol), Kathon C G (methyl and methylchloroisothiazolinone), parabens (methyl, ethyl, propyl, butyl hydrobenzoates), phenoxyethanol(2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion and/or suspension vaccine may further comprise at least one pH adjuster. Suitable pH adjusters in the nanoemulsion and/or suspension vaccine of the disclosure include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion and/or suspension vaccine can comprise a chelating agent. In one embodiment of the disclosure, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion and/or suspension vaccine can comprise a buffering agent, such as a pharmaceutically acceptable buffering agent. Examples of buffering agents include, but are not limited to, 2-Amino-2-methyl-1,3-propanediol, 2-Amino-2-methyl-1-propanol, L-(+)-Tartaric acid, ACES, ADA, Acetic acid, Ammonium acetate solution, Ammonium bicarbonate, Ammonium citrate dibasic, Ammonium formate solution, Ammonium oxalate monohydrate, Ammonium phosphate dibasic solution, Ammonium phosphate monobasic solution, Ammonium sodium phosphate dibasic tetrahydrate, Ammonium sulfate solution, Ammonium tartrate dibasic solution, BES buffered saline, BICINE buffer Solution, Bicarbonate buffer solution, Boric acid, Calcium acetate hydrate, Calcium carbonate, Calcium citrate tribasic tetrahydrate, Citrate Concentrated Solution, Citric acid, Diethanolamine, Ethylenediaminetetraacetic acid disodium salt dihydrate, Formic acid solution, Glycine, HEPES buffered salinelmidazole buffer Solution, Imidazole, Lipoprotein Refolding Buffer, Lithium acetate dihydrate, Lithium citrate tribasic tetrahydrate, MES monohydrate, MOPS, Magnesium acetate solution, Magnesium citrate tribasic nonahydrate, Magnesium formate solution, Magnesium phosphate dibasic trihydrate, PIPES, Phosphate buffered saline, Potassium bicarbonate, Potassium chloride, Potassium citrate monobasic, Potassium citrate tribasic solution, Potassium formate, Potassium oxalate monohydrate, Potassium phosphate dibasic, Potassium phosphate monobasic, Sodium acetate, Sodium bitartrate monohydrate, Sodium carbonate decahydrate, Sodium carbonate, Sodium oxalate, TRIS Glycine buffer solution, TRIS acetate-EDTA buffer solution, TRIS buffered saline, TRIS glycine SDS buffer solution.

The nanoemulsion and/or suspension vaccine can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the disclosure feature nanoemulsion and/or suspension vaccines that may readily be diluted with water or another aqueous phase to a desired concentration without impairing their desired properties.

The nanoemulsions of the disclosure can be formed using classic emulsion forming techniques. See e.g., U.S. 2004/0043041. In an exemplary method, the non-polar composition (e.g., oil) comprising the adjuvants is mixed with the aqueous phase under relatively high shear forces (e.g., using high hydraulic and mechanical forces) to obtain a nanoemulsion comprising oil droplets having an average diameter of less than about 1000 nm. Some embodiments of the disclosure employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In one embodiment, the nanoemulsions used in the methods of the disclosure comprise droplets of an oily discontinuous phase dispersed in an aqueous continuous phase, such as water or PBS. The nanoemulsions of the disclosure are stable, and do not deteriorate even after long storage periods. Certain nanoemulsions of the disclosure are non-toxic and safe when swallowed, inhaled, or contacted to the skin of a subject.

The compositions of the disclosure can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The disclosure provides a multi-component vaccine. In one embodiment, the disclosure provides a first component comprising a TLR-adjuvant formulation comprising a TLR4 agonist and a TLR7 agonist in a non-polar buffer. In a further embodiment, the TLR7 agonist comprises a structure of Formula I, I(a), or I(b) as described above. In still a further embodiment, the TLR4 agonist comprises a structure of Formula II, II(a), II(b) or V. In another embodiment, the TLR4 and TLR7 agonists are linked and comprise a structure of formula III or IV(b). In another embodiment, the disclosure provides a second component comprising an antigen/immunogen in a polar buffer. In one embodiment, the antigen/immunogen is any water soluble antigen/immunogen to be delivered to a subject to induce an immune response. In a further embodiment, the antigen/immunogen can be from a bacteria, virus, fungus, protozoa, cancer cell etc.

Examples of viral antigens include, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens.

Examples of bacterial antigens that can be used include, but are not limited to, pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; small pox; allergies and cancers.

Fungal antigens for use with compositions and methods of the disclosure include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Protozoal and other parasitic antigens for use in the methods and compositions of the disclosure include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

Specific non-limiting examples of tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17(gp100), GnT-V intron V sequence (N-acetylglucoaminyltransferase V intron V sequence), Prostate Ca psm, PRAME (melanoma antigen), beta-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In addition, the immunogenic molecule can be an autoantigen involved in the initiation and/or propagation of an autoimmune disease, the pathology of which is largely due to the activity of antibodies specific for a molecule expressed by the relevant target organ, tissue, or cells, e.g., SLE or MG. In such diseases, it can be desirable to direct an ongoing antibody-mediated (i.e., a Th2-type) immune response to the relevant autoantigen towards a cellular (i.e., a Th1-type) immune response. Alternatively, it can be desirable to prevent onset of or decrease the level of a Th2 response to the autoantigen in a subject not having, but who is suspected of being susceptible to, the relevant autoimmune disease by prophylactically inducing a Th1 response to the appropriate autoantigen. Autoantigens of interest include, without limitation: (a) with respect to SLE, the Smith protein, RNP ribonucleoprotein, and the SS-A and SS-B proteins; and (b) with respect to MG, the acetylcholine receptor. Examples of other miscellaneous antigens involved in one or more types of autoimmune response include, e.g., endogenous hormones such as luteinizing hormone, follicular stimulating hormone, testosterone, growth hormone, prolactin, and other hormones.

In a specific embodiment, the antigen/immunogen is an HA-stalk domain (aka HA-stem domain) antigen of Influenza. The antigen can comprise from 6 or more amino acids so long as they are antigenic. In some embodiments, the antigen can be two peptides derived from the HA stalk domain. In some embodiments, the peptides are linked by a linker to form a multimer. In another embodiment, the antigen used in the methods and compositions of the disclosure comprises a plurality of antigenic peptide or polypeptides derived from the stalk domain. In one embodiment, the immune response is raised against any peptide of 6 or more amino acids from the “Stem domain” column of Table 1. Typically a peptide from the stem domain that is highly conserved across the influenza variants/strains is used to improve an immune response to multiple strains of influenza. Exemplary sequences of the stalk/stem domain that can be used as antigens are set forth in Table 1:

TABLE 1
Exemplary Influenza A Hemagglutinin Sequences

| HA2 domain | | | | |
|---|---|---|---|---|
| Subtype | | | Trans- | cyto- |
| (Genbank | Stem | Luminal | membrane | plasmic |
| No.) | domain | domain | domain | domain |
| H1 | GLFGAIGFIEGGWT | MGIYQ | ILAIYSVASSL | NGSLQCRICI |
| PR8-H1N1 | GMIDGYGYHHQNE | | VLLVSLGAISF | CI |
| (EF467821.1) | QGSGYAADQKSTQN | | WMCS | |
| | AINGITNKVNTVIEK | | | |
| | MNIQFTAVGKEFNKL | | | |
| | EKRMENLNKKVDDG | | | |
| | FLDIWTYNAELLVLL | | | |
| | ENERTLDFHDSNVKN | | | |
| | LYEKVKSQLKNNAK | | | |
| | EIGNGCFEFYHKCDN | | | |
| | ECMESVRNGTYDYP | | | |
| | KYSEESKLNREKVDG | | | |
| | VKLES | | | |
| H2 | GLFGAIAGFIEOGWQ | MGVYQ | ILAIYATVAGSL | NGSLQCRICI |
| (L11136) | GMIDGWYGYHHSND | | SLAIMIAGISLW | CI |
| | QGSGYAADKESTQK | | MCS | |
| | AIDGITNRVNSVIEK | | | |
| | MNTQFEAVGKEFSNL | | | |
| | EKRLENLNKKMEDO | | | |
| | FLDVWTYNAELLVL | | | |
| | MENERTLDFHDSNV | | | |
| | KNLYDRVRMQLRDN | | | |
| | AKELGNGCFEFYHKC | | | |
| | DDECMNSVKNGTYD | | | |
| | YPKYEEESKLNRNEI | | | |
| | KGVKLSN | | | |
| H3 | GLFGAIAGFIENGWE | SGYKD | WILWISPAISCP | RGNIRCNICI |
| HK68-H3N2 | GMIDGWGFRHQNS | | LLCVVLLGFIM | CI |
| (EF409245) | EGTGQAADLKSTQA | | WACQ | |
| PDB: 1HGJ | AIDQINGKLNRVIEKT | | | |
| | NEKFHQIEKEFSEVE | | | |
| | GRIQDLEKYVEDTKI | | | |
| | DLWSYNAELLVALE | | | |
| | NQHTIDLTDSEMNKL | | | |
| | FEKTRRQLRENAED | | | |
| | MGNGCFKIYWKCDN | | | |
| | ACIESIRNGTYDHDV | | | |
| | YRDEALNNRFQIKGV | | | |
| | ELK | | | |
| H4 | GLFGAIAGFIENGWQ | QGYKD | IILWISPSISCPLL | NGNIRCQICI |
| (D90302) | GLIDGWYGPRHQNA | | VALLLAFILWA | CI |
| | EGTGTAADLKSTQA | | CQ | |
| | AIDQINGKLNRLIEKT | | | |
| | NDKYHQIEKEFEQVE | | | |
| | GRIQDLENYVEDTKI | | | |
| | DLWSYNAELLVALE | | | |
| | NQHTIDVTDSEMNKL | | | |
| | FERVRRQLRENAEDK | | | |
| | GNGCPEIFHKCDNNC | | | |
| | IESIRNGTYDHDIYRD | | | |
| | EAINNRFQIQGVKLT | | | |
| H5 | GLFGAIAFIEGGWQ | MGVYQ | ILSIYSTVASSL | NGSLQCRICI |
| (X07826) | GMVDGWYGYHHSN | | ALAIMIAGLSP | CI |
| | EQGSGYAADKESTQ | | WMCS | |
| | KAIDGITNKVNSIIDK | | | |
| | MNTRPEAVGKEEFNN | | | |
| | LERRVENLNKKMED | | | |
| | GFLDVWTVNVELLV | | | |
| | LMENERTLDFHDSNV | | | |
| | NNLYDKVRLQLKDN | | | |
| | ARELGNGCPEPYHKC | | | |
| | DNECMESVRNGTYD | | | |
| | YPQYSEEARLNREEIS | | | |
| | GVKLES | | | |
| H6 | GLFGAIGFIEGGWT | LGVYQ | ILAIYSTVSSSL | NGSMQCRICI |
| (D90303) | GMIDGWYGHHENS | | VLVGLIIAVGL | ICI |
| | QGSGYAADRESTQR | | WMCS | |
| | AVDGITNKVNSIIDK | | | |
| | MNTQPEAVDHEFSNL | | | |
| | ERRIDNLNKRMEDGF | | | |
| | LDVWTVNAELLVLL | | | |
| | ENERTLDLHDANVK | | | |
| | NLYERVKSQLRDNA | | | |
| | MILGNGCPEPWMKC | | | |
| | DDECMESVKNGTYD | | | |
| | YPKYQDESKLNRQEI | | | |
| | ESVKLES | | | |
| H7 | GLFGAIGFIENGWE | SGYKD | VILWPSPGASCF | NGNMRCTICI |
| (M24457) | GLVDGWYGPRHQNA | | LLLAIAMGLVFI | ICI |
| | QGEGTAADYKSTQS | | CVK | |
| | AIDQITGKLNRLIEKT | | | |
| | NQQPELIDNEFTEVE | | | |
| | KQIGNLINWTKDSITE | | | |
| | VWSYNAELIVAMEN | | | |
| | QHTIDLADSEMNRLY | | | |
| | ERVRKQLRENAEED | | | |
| | GEGPEIFHKCDDDC | | | |
| | MASIRNNTYDHSKYR | | | |
| | EEAMQNRIQIDPVKLS | | | |
| H8 | GLFGAIGFIEGGWS | NTTYK | ILSIYSTVAASL | NGSCRCMFCI |
| (D90304) | GMIDGWYGFHHSNS | | CLAILIAGGLIL | FCI |
| | EGTGMAADQKSTQE | | GMQ | |
| | AIDKITNKVNNIVDK | | | |
| | MNREFEVVNHEFSEV | | | |
| | EKRINMINDKIDDQIE | | | |
| | DLWAYNAELLVLLE | | | |
| | NQETLDEHDSNVKN | | | |
| | LPDEVKRRLSANAID | | | |
| | AGNCFDILHKCDNE | | | |
| | CMETIKNGTYDHKE | | | |
| | YEEEAKLERSKINGV | | | |
| | KLEE | | | |
| H9 | GLFGAIAGFIEGGWP | EGTYK | ILTIYSTVASSL | NGSCRCNICI |
| (D90305) | GLVAGWYGFQNSND | | VLAMGFAAFLF | CI |
| | QGVGMAADKGSTQK | | WAMS | |
| | AIDKITSKVNNIIDKM | | | |
| | NKQYEVIDHEFNELE | | | |
| | ARLNMINNKIDDQIQ | | | |
| | DIWAYNAELLVLLEN | | | |
| | QKTLDEHDANVNNL | | | |
| | YNKVKRALGSNAVE | | | |
| | DGNGCFELVHKCDD | | | |
| | QCMETIRNGTYDRQ | | | |
| | KYQEESRLERQKIEG | | | |
| | VKLES | | | |
| H10 | GLFGAIGFIENGWE | SGYKD | IILWFSFGESCF | NFNMRCTICI |
| (M21647) | GMVDGWYGFRHQN | | VLLAVVMGLV | ICI |
| | AQGTQAADYKSTQ | | FFCLK | |
| | AAIDQITGKLNRLIEK | | | |
| | TNTEFESIESEFSETEH | | | |
| | QIGNVINWTKDSITDI | | | |
| | WTYNAELLVAMENQ | | | |
| | HTIDMADSEMLNLYE | | | |
| | RVRKQLRQNAEEDG | | | |
| | EGCFEIYHTCDDSCM | | | |
| | ESIRNNTYDHSQVRR | | | |
| | EALLNRLNINPVKLS | | | |
| | [SEQ ID NO.: 91] | | | |
| H11 | GLFGAIAGFIEGGWP | GNVYK | ILSIYSCIASSLV | NGSCRCTICI |
| (D90306) | GLINGWYGFQHRDE | | LAALIMGFMFW | CI |
| | EGTGIAADKESTQKA | | ACS | |
| | IDQITSKVNNIVDRM | | | |
| | NTNFESVQHEFSEIEE | | | |
| | RINQLSKHVDDSVVD | | | |
| | IWSYNAQLLVLLENE | | | |
| | ETLDLHDSNVRNLHE | | | |
| | KVRRMLKDNAKDEG | | | |
| | NGCPTPYHKCDNKCI | | | |
| | ERVRNGTYDHKEFEE | | | |
| | ESKINRQEIEGVKLDSS | | | |
| | [SEQ ID NO.: 92] | | | |
| H12 | GLFGAIAGFIEGGWP | NSTYK | ILSIYSSVASSLV | GNVRCTFCI |
| (D90307) | GLVAGWYGFQHQNA | | LLLMIIGGFIFG | CI |
| | EGTGIAADRDSTQRA | | CQN | |
| | IDNMQNKLNNVIDK | | | |
| | MNKQFEVVNHEFSE | | | |
| | VESRINMINSKIDDQI | | | |
| | TDIWAYNAELLVLLE | | | |
| | NQKTLDEHDANVRN | | | |
| | LHDRVRRVLRENAID | | | |
| | TGDGCFEILHKCDNN | | | |
| | CMDTIRNGTYNHKE | | | |
| | YEEESKIBRQKVNGV | | | |
| | KLEE | | | |
| H13 | GLFGAIAGFIEGGWP | DNVYK | ALSIYSCIASSV | GNCRFNV |
| (D90308) | GLINGWYGFQHQNE | | VLVGLILSFIM | CI |
| | QGTGIAADKESTQKA | | WACSS | |
| | IDQITTKINNIIDKMN | | | |
| | GNYDSIRGEFNQVEK | | | |
| | RINMLADRIDDAVTD | | | |
| | IWSYNAKLLVLLEND | | | |
| | KTLDMHDANVKNLH | | | |
| | EQVRRELKDNAIDEG | | | |
| | NGCFELLHKCNDSC | | | |
| | METIRNGTYDHTEYA | | | |
| | EESKLKRQEIDGILKL | | | |
| | SE | | | |
| H14 | GLFGAIAGFIENGWQ | MGYKD | IILWISFSMSCF | NGNIRCQICI |
| (M35997) | GLIDGWYGFRHQNA | | VFVALILGFVL | CI |
| | EGTGTAADLKSTQA | | WACQ | |
| | AIDQINGKLNRLIEKT | | | |
| | NEKYHQIEKEFEQVE | | | |
| | GRIQDLEKYVEDTKI | | | |
| | DLWSYNAELLVALE | | | |
| | NQHTIDVTDSEMNKL | | | |
| | PERVRRQLRENAEDQ | | | |
| | GNGCFEIFHQCDNNC | | | |
| | IESIRNGTYDHNIYRD | | | |
| | EAINMRIKINPVTLT | | | |
| H15 | GLFGAIAGFIENGWE | SGYKD | VILWFSFGASC | GNLRCTICI |
| (L43917) | GLIDGWYGFRHQNA | | VMLLAIAMGLI | |
| | QGQGTAADYKSTQA | | PMCVKN | |
| | AIDQITGKLNRLIEKT | | | |
| | NKQFELIDNEFTEVE | | | |
| | QQIGNVINWTRDSLT | | | |
| | EIWSYNAELLVAME | | | |
| | NQHTIDLADSEMNKL | | | |
| | YERVRRQLRENAEED | | | |
| | GEGCFEIFHRCDDQC | | | |
| | MESIRNNTYNHTEYR | | | |
| | QEALQNRIMINPVKLS | | | |
| H16 | GLFGAIAGPIEOGWP | DNVYK | VLSIYSCIASSIV | NGSCRFNV |
| (EU293865) | GLINGWYGFQHQNE | | LVGLILAPIMW | |
| | QGTGIAADKASTQKA | | ACS | |
| | INEITTKINNIIEKMNG | | | |
| | NYDSIRGEFNQVEKR | | | |
| | INMLADRVDDAVTDI | | | |
| | WSYNAKLLVLLEND | | | |
| | ETLDLHDANVRNLH | | | |
| | DQVKRALKSNAIDEG | | | |
| | DGCFNLLHFCNDSC | | | |
| | METIRNGTYNHEDYR | | | |
| | EESQLKRQEIEGIKLK | | | |
| | TE | | | |

In one embodiment, the nanoemulsion and/or suspension vaccine can comprise about 0.001 μg to about 90 μg of each influenza antigen strain, per dose. In a further embodiment, the nanoemulsion and/or suspension vaccine can comprise about 15 μg or less influenza strain, per dose. In another embodiment, the nanoemulsion and/or suspension vaccine can comprise more than one influenza immunogen.

In another embodiment, the disclosure provides a mixture or nanoemulsion comprising a TLR4 agonist, a TLR7 agonist and an antigen/immunogen. In a further embodiment, the TLR7 agonist comprises a structure of Formula I, I(a), or I(b) as described above. In still a further embodiment, the TLR4 agonist comprises a structure of Formula II, II(a), II(b) or V. In another embodiment, the TLR4 and TLR7 agonists are linked and comprise a structure of formula III or IV(b).

In one embodiment, a vaccine of the disclosure is delivered to a subject as a nanoemulsion and/or suspension.

The nanoemulsion and/or suspension compositions of the disclosure function as a vaccine containing a TLR4 agonist and TLR7 agonist adjuvants in combination with an antigen/immunogen. Adjuvants serve to: (1) bring the antigen/immunogen into contact with the immune system and influence the type of immunity produced, as well as the quality of the immune response (magnitude or duration); (2) decrease the toxicity of certain antigens/immunogens; (3) reduce the amount of antigen/immunogen needed for a protective response; (4) reduce the number of doses required for protection; (5) provide greater cross-reactivity and protection (e.g., to various influenza strains); (6) enhance immunity in poorly responding subsets of the population and/or (7) provide solubility to some vaccines components.

The methods comprise administering to a subject a nanoemulsion vaccine, wherein the nanoemulsion vaccine comprises droplets having an average diameter of less than about 1000 nm. The nanoemulsion vaccine further comprises (a) an aqueous phase, (b) at least one oil, (c) at least one surfactant, (d) at least one organic solvent, (e) at least one immunogen (e.g., an influenza HA-stalk/stem derived antigen), (f) a TLR4 and TLR7 agonist; and (g) optionally comprising at least one chelating agent, or any combination thereof.

The human or animal subject can produce a protective immune response after at least one administration of the nanoemulsion and/or suspension vaccine. In one embodiment, the subject undergoes seroconversion after a single administration of the nanoemulsion and/or suspension vaccine. In a further embodiment, the subject is selected from adults, elderly subjects, juvenile subjects, infants, high risk subjects, pregnant women, and immunocompromised subjects. In another embodiment, the nanoemulsion and/or suspension vaccine may be administered intranasally.

The nanoemulsion and/or suspension vaccines of the disclosure can surprisingly stimulate the immune response utilizing less antigen than is required by currently used vaccines. Further, vaccines comprising the nanoemulsion and/or suspensions of the disclosure require fewer administrations and generate stronger responses in subjects.

The nanoemulsion and/or suspension vaccine adjuvant can be combined with an antigen or the nanoemulsion and/or suspension vaccine adjuvant can be sequentially administered with an antigen. Alternatively, or in combination, the nanoemulsion and/or suspension vaccine adjuvant can be administered to a subject having exposure to an antigen (i.e., prophylactic exposure, environmental exposure, etc.). Thus, in a method of the disclosure, the nanoemulsion and/or suspension vaccine adjuvant can comprise at least one immunogen (e.g., an influenza immunogen), or the nanoemulsion and/or suspension can be sequentially administered with one or more immunogens (e.g., one or more influenza immunogen).

The TLR4 agonist and TLR7 agonist can be combined with other vaccine therapies. For example, the nanoemulsion and/or suspension of the disclosure may be combined with one or more commercial influenza vaccines, such as FLUVIRIN and FLUZONE, or the nanoemulsion and/or suspension may be sequentially administered with one or more commercial influenza vaccines.

The nanoemulsion and/or suspension vaccine of the disclosure can be administered to a subject which has not previously received a specific vaccine (e.g., an influenza vaccine), and the nanoemulsion and/or suspension vaccine can be administered to a subject who had previously received a vaccine comprising a same or similar immunogen (e.g., an influenza vaccine).

For influenza vaccinations, the nanoemulsion and/or suspension vaccine can be given in at least a single administration annually to address seasonal influenza, pandemic flu, or a combination thereof. At least one administration of the nanoemulsion and/or suspension vaccine can be given to provide sustained protection, or more than one administration of the nanoemulsion and/or suspension vaccine can be given to provide sustained protection.

The composition (e.g., the nanoemulsion and/or suspension) can be applied using any pharmaceutically acceptable method, such as for example, intranasal, buccal, sublingual, oral, rectal, ocular, parenteral (intravenously, intradermally, intramuscularly, subcutaneously, intracisternally, intraperitoneally), pulmonary, intravaginal, locally administered, topically administered, topically administered after scarification, mucosally administered, via an aerosol, or via a buccal or nasal spray formulation. Further, the nanoemulsion and/or suspension vaccine can be formulated into any pharmaceutically acceptable dosage form, such as a liquid dispersion, gel, aerosol, pulmonary aerosol, nasal aerosol, ointment, cream, semi-solid dosage form, and a suspension.

The terms “pharmaceutically acceptable” or “pharmacologically acceptable,” as used herein, refer to compositions that do not substantially produce adverse allergic or adverse immunological reactions when administered to a host (e.g., an animal or a human).

As used herein, the term “intranasal(ly)” refers to application of the compositions of the disclosure to the surface of the skin and mucosal cells and tissues of the nasal passages, e.g., nasal mucosa, sinus cavity, nasal turbinates, or other tissues and cells which line the nasal passages.

As used herein, the term “topical(ly)” refers to application of the compositions of the disclosure to the surface of a tissue (e.g., buccal, lingual, sublingual, masticatory, respiratory or nasal mucosa, nasal turbinates and other tissues and cells which line hollow organs or body cavities).

The disclosure contemplates that many variations of the described nanoemulsion and/or suspensions will be useful in the methods of the disclosure.

The nanoemulsion and/or suspensions can be delivered (e.g., to a subject or customers) in any suitable container. Suitable containers can be used that provide one or more single use or multi-use dosages of the nanoemulsion and/or suspension for the desired application. In some embodiments of the disclosure, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

In another embodiment, the components (i.e., the oil-phase or solubilized adjuvant mixture comprising the TLR-4 and -7 agonists, and the aqueous phase comprising an immunogen) are provided separately and subsequently formed into a nanoemulsion or a suspension by a technician or physician.

For example, formulations would comprise TLR4- and TLR7-agonists (e.g., compounds of formula I and II) dissolved in a clear solution of mixed solvents that are “saturated” in the sense that when any aqueous solution is added, a suspension would form and that suspension would be syringeable to allow for i.m. administration. The aqueous solution to be added to the clear solution of adjuvant would contain the antigen of choice and both components are designed to be mixed in 1:1 proportions to form the suspension for injection.

These nanoemulsion-containing or adjuvant mixture-containing containers can further be packaged with instructions for use to form kits.

In some embodiments the TLR agonist will comprise a whole virus or microorganism which may be engineered to express a desired antigen in combination with a TLR agonist. In some embodiments the microorganism or virus which functions as a TLR agonist may be genetically engineered to express an HA stalk antigen in a single microbial or viral vehicle thereby facilitating administration to a host having a condition wherein enhanced antigen specific cellular immune response are desirably elicited. The TLR agonism for a particular compound may be assessed in any suitable manner.

A bond indicated by a straight line and a dashed line indicates a bond that may be a single covalent bond or alternatively a double covalent bond. But in the case where an atom's maximum valence would be exceeded by forming a double covalent bond, then the bond would be a single covalent bond.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, npropyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the disclosure. A “lower alkyl” or “lower alkylene” is a shorter chain alkyl or alkylene group, generally having eight or fewer carbon atoms.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to 15 multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxyl)propyl, and the like).

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples ofheterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

As used herein, the terms “heteroatom” or “ring heteroatom” are meant to include oxygen (O), nitrogen (N), sulfur (S), phosphorus (P), and silicon (Si).

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR′—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the disclosure includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)z, fluoro (C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the disclosure includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where sand dare independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

A “substituent group,” as used herein, means a group selected from the following moieties: (A) —OH, —NH₂, —SH, —CN, —CF₃, —CCl₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C1-C20 alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

The pharmaceutically acceptable salts of the compounds useful in the disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, Pa., p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. In some embodiments, a patient is human.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof.

The term “antibody” or “antibodies” as used herein refers to all types of immunoglobulins, including IgG, IgM, IgA, IgD, and IgE, and any sub-isotype, including IgG1, IgG2a, IgG2b, IgG2c, IgG3 and IgG4, IgE1, IgE2, etc., and may include Fab or antigen-recognition fragments thereof. The antibodies may be monoclonal or polyclonal and may be of any species of origin, including e.g., mouse, rat, rabbit, horse, or human, or may be chimeric antibodies. See, e.g., M. Walker et al., Molec. Immunol. 1989, 26:403-11; Morrision et al., Proc. Nat'l. Acad. Sci., 1984, 81:6851; Neuberger et al, Nature, 1984, 312:604. The antibodies may be recombinant monoclonal antibodies produced according to the methods disclosed in U.S. Pat. No. 4,474,893 (Reading) or U.S. Pat. No. 4,816,567 (Cabilly et al.). The antibodies may also be 15 chemically constructed by specific antibodies made according to the method disclosed in U.S. Pat. No. 4,676,980 (Segel et al).

The term “antigen” refers, in the usual and customary sense, to a substance that binds specifically to an antibody or that can be recognized by antigen receptors (e.g., B-cell receptor, T-cell receptor and the like) of the adaptive immune system.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound with structure of Formula (I) and (II) as disclosed herein, including embodiments thereof. The term “modulating” as used herein refers to either increasing or decreasing the level of activity of the modulated entity, e.g., immune response. Accordingly, an immune response can be activated or deactivated as a consequence of administration of an agent, e.g., a compound with structure of Formula (I) as disclosed herein, including embodiments thereof. The term “activated” means an enhancement in the activity of the activated entity. The term “deactivated” means a diminution in the activity of the deactivated entity. In some embodiment, a deactivated immune response is nonetheless measurable, albeit at a reduced level compared to levels absent deactivation.

The working examples below are provided to illustrate, not limit, the invention. Various parameters of the scientific methods employed in these examples are described in detail below and provide guidance for practicing the invention in general.

### EXAMPLES

### Animals

Animal experiments performed at the University of California San Diego, La Jolla, Calif., USA (UCSD) were approved by the UCSD Institutional Animal Care and Use Committee (IACUC) (S09331), and animal experiments performed at the Icahn School of Medicine were approved by the Mount Sinai IACUC (LA13-00084). Seven- to 9-week-old C57BL/6(wild-type [WT]) and OT-1 transgenic mice in which the CD8^{L} T cells express a T cell receptor specific for the ovalbumin (OVA257-264) peptide (C57BL/6 background) were purchased from the Jackson Laboratories (Bar Harbor, Mass.). TrifLps2/Lps2 mice are described in Hoebe et al., Nature 424:743-748, 2003. Tlr4^{-/-} and Myd88^{-/-} mice were a gift from Shizuo Akira (Osaka University, Osaka, Japan). These strains were backcrossed for 10 generations onto the C57BL/6 background at the University of California, San Diego. For immunization with influenza virus antigens, 6- to 8-week-old WT female BALB/c mice were purchased from Jackson Laboratories (Bar Harbor, Mass.). The animals were anesthetized with ketamine/xylazine before intranasal viral infection.

### Reagents

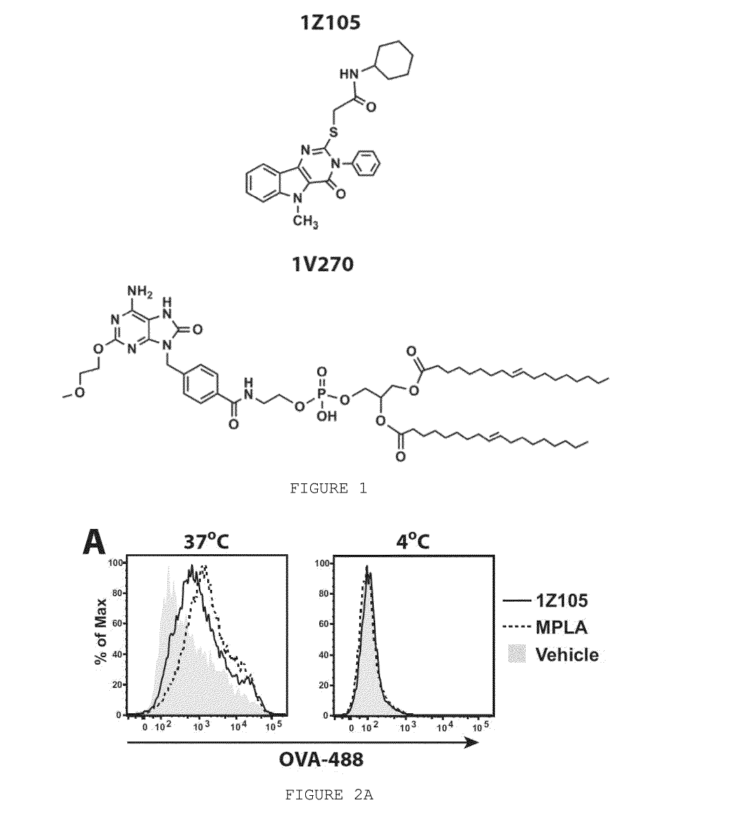

Compounds 1Z105 and 1V270 (FIG. 1) were synthesized in the laboratory as previously described (Chan et al., J. Med. Chem. 56:4206-4223, 2013; Chan et al., Bioconjug. Chem., 20:1194-1200, 2009), dissolved in dimethyl sulfoxide (DMSO) (Sigma-Aldrich, St. Louis, Mo.) as 20 to 100 mM stock solutions, and stored at −20° C. The endotoxin levels of these drugs were determined by Endosafe (Charles River Laboratory, Wilmington, Mass., USA) and were less than 10 endotoxin units (EU)/μmol. MPLA and AddaVax were purchased from Invivogen (San Diego, Calif.). OVA (endotoxin levels, 12 to 18 EU/mg protein) was purchased from Worthington Biochemical Co. (Lakewood, N.J.).

The recombinant hemagglutinin (rHA) antigens derived from influenza viruses A/Puerto Rico/8/1934 (H1N1) (PR/8) and A/California/04/2009 (H1N1) (Cal/09) and the chimeric HAs (cHAs) (cH2/1PR/8 and cH6/1PR/8) were expressed from baculovirus in High Five cells in the laboratory as soluble trimers utilizing the T4 phage fibritin natural trimerization domain and a C-terminal 6× His tag for purification as previously described (29). Both chimeric HAs utilize the PR/8 stalk, as previously described, and their globular heads are derived from A/Singapore/1-MA12E/1957 (H2N2) and A/mallard/Sweden/81/2002 (H6N1) (Krammer et al., J. Virol., 86:5774-5781, 2012). These proteins were purified with Ni-nitrilotriacetic acid (NTA) agarose (Qiagen, Hilden, Germany). A second version of recombinant PR/8 and Cal/09 HAs utilizing a streptomycin (Strep) tag purification domain and the GCN4 leucine zipper as a trimerization domain was expressed in SF9. Cells and purified with a StrepTactin Sepharose column (GE Healthcare Life Sciences, Pittsburgh, Pa.). Recombinant HA from A/Vietenam/1203/2004 (H5N1) was purchased from Sino Biologicals (Beijing, China). The cH5/3 construct incorporates the HA globular head of A/Vietenam/1203/2004 and the HA stalk domain of A/Perth/16/2009. The virus was rescued as a “6-plus-2” reassortant utilizing an N3 subtype neuraminidase derived from A/swine/Missouri/4296424/2006 in the PR/8 background. The 2009-2010 formulation of Fluzone was manufactured by Sanofi-Pasteur. The influenza viruses were all grown in 8- to 10-day-old embryonated chicken eggs. Viruses used as enzyme-linked immunosorbent assay (ELISA) substrates were subsequently purified and concentrated by centrifugation through a 30% sucrose gradient.

### Formulation

In initial studies, immunostimulatory effects and cytotoxicity of eight formulations of adjuvant (EV1-8) without antigens were evaluated by IL-6 induction and MTT assay using bone marrow derived dendritic cells (BMDCs). BMDCs were incubated with 1 or 5% EV1, 2, 3, 4, 5, 6, 7 and 8 overnight. EV1, EV5, and EV6 showed cytotoxicity at 1% concentration. At 5% concentration, all except EV7 were cytotoxic. None of the formulations induced IL-6 release by BMDCs. These data indicate that the cytotoxic effects were not correlated to immunostimulatory effects measured by IL-6 induction. Based on the concentration of 1Z105 and 1V270 used in previous experiments to test adjuvant activities in murine influenza virus infection models, concentrations of 1.8-3.6 mg/mL for 1Z105 or 1Z204 and 0.22-0.44 mg/mL for 1V270 were prepared. The solubility of 1V270 in EV3 and EV7 was achieved to the requested concentration.

Immunostimulatory potencies of formulated 1Z105, and 1V270 were then tested in BMDCs in vitro. BMDCs were stimulated with serially diluted formulated compounds overnight. IL-6 release in the culture supernatants were measured by ELISA. EV6-formulated 1Z105 induced significantly lower IL-6 compared to DMSO formulated 1Z105. In contrast, EV3 or EV7 formulated 1V270 achieved similar maximum induction of IL-6, however, the potency of formulated 1V270 was about one log lower than DMSO-formulated 1V270. Significantly reduced IL-6 release by EV7-formulated 1V270 was detected at the highest concentration (10 μM).

### In Vivo Immunization Studies Using Model Antigen and Influenza Virus Antigens

C57BL/6 WT, Myd88−/−, or TrifLps2/Lps2 mice were i.m. immunized with 20 μg of OVA with 1Z105 (89.4 μg/dose, equivalent to 200 nmol/animal), 1V270 (10.8 μg/dose, equivalent to 10 nmol/animal), or a combination of 1Z105 (89.4 μg/dose) and 1V270 (10.8 μg/dose) in a total volume of 50 μl on days 0 and 14. Vehicle (10% DMSO in saline) and AddaVax (1:1 ratio with antigen in saline) were used as controls. WT BALB/c mice were i.m. immunized with rHA (5 μg per mouse for rPR/8, rCal/09, cH2/1, and cH6/1 HAs and 2 μg per mouse for rVN/04 HA) plus 1Z105 (89.4 μg/dose), 1V270 (10.8 μg/dose), or a combination of 1Z105 (89.4 μg/dose) and 1V270 (10.8 μg/dose) in 10% DMSO-phosphatebuffered saline (PBS). AddaVax was used at a 1:1 ratio with antigen in PBS. The control groups received antigen in 10% DMSO-PBS (no adjuvant), a combination of 1Z105 (89.4 μg/dose) and 1V270 (10.8 μg/dose) in 10% DMSO-PBS without antigen (adjuvant only), or the vehicle. Fluzone was used at 50 ng/HA per mouse, and all immunizations were delivered into the gastrocnemius muscle in a total volume of 50 μl.

### Alignment of Influenza Virus HA Protein Sequences Utilized in this Study

HA protein sequences in vaccination and challenge strains relevant to this work were aligned to generate a phylogenetic tree with MEGA5. The Caption Expert tool in Mega 5.1 was used as follows. The evolutionary history was inferred using the neighbor-joining method. The optimal tree with the sum of branch lengths equaling 2.82138273 is shown. The percentage of replicate trees in which the associated taxa clustered together in the bootstrap test (1,000 replicates) are shown next to the branches. The tree is drawn to scale, with branch lengths in the same units as those of the evolutionary distances used to infer the phylogenetic tree. The evolutionary distances were computed using the Poisson correction method are in units of the number of amino acid substitutions per site. The analysis involved 11 amino acid sequences. All positions containing gaps and missing data were eliminated. There were a total of 545 positions in the final data set. Evolutionary analyses were conducted in MEGA5.

### In Vitro Assay for Antigen Uptake and Costimulatory-Molecule Expression in Mouse Bone Marrow-Derived Dendritic Cells

Mouse bone marrow-derived dendritic cells (mBMDCs) were prepared from wild type or Tlr4^{-/-} C57BL/6 mice. mBMDCs (10⁵ cells per well) were plated in 96-well plates in 200 μl of complete RPMI 1640. The cells were incubated with 10 μM 1Z105 or vehicle for 18 h at 37° C., 5% CO2; 10 μg/ml OVA conjugated with Alexa Fluor 488 (Life Technologies) was added to the culture during the last 30 min of incubation. Cells incubated at 4° C. served as negative controls. MPLA (1 μg/ml) was used as a positive control. In studies for expression of costimulatory molecules, mBMDCs were incubated with 1Z105 (2 or 10 μM) or MPLA (0.04 μg/ml) overnight and stained for surface expression of CD40 and CD86. The cells were stained for CD11c, and OVA uptake and expression of CD40 and CD86 in the CD11chi-gated population was determined by flow cytometry.

### Cytokine Induction of Human Monocyte-Derived Dendritic Cells

Human primary dendritic cells (DCs) were generated. Briefly, CD14⁺ cells were isolated from buffy coats of healthy human donors (New York Blood Center) with anti-human CD14 antibody-labeled magnetic beads and iron-based MiniMACS liquid separation columns (Miltenyi Biotec, San Diego, Calif.). For the generation of immature DCs, CD14⁺ cells were incubated at 37° C. for 5 days in complete RPMI 1640 supplemented with 500 U/ml human granulocyte-macrophage colony-stimulating factor (hGM-CSF) and 1,000 U/ml human interleukin 4 (hIL-4) (Peprotech, Rocky Hill, N.J.). DCs were incubated with 10 μM 1Z105, 50 ng/ml of lipopolysaccharide (LPS) (L2654, Escherichia coli 026:B6; Sigma-Aldrich), or vehicle (0.5% DMSO in medium) for 18 h. The supernatants were stored at −20° C. for later quantification of released cytokines. Quantification of IL-1β, IL-6, IL-8, IL-12p70, and tumor necrosis factor alpha (TNF-α) released in supernatants was performed using the Milliplex Multiplex Assays (Luminex; Millipore, Billerica, Mass.) according to the manufacturer's instructions. Data were analyzed using the Milliplex Analyst software (Millipore).

### OT-1 CD8 T Cell Proliferation Assay

Naive CD8⁺ T cells from OT-1 C57BL/6 mice were isolated using an EasySep Mouse CD8⁺ T Cell Isolation Kit and stained with carboxyfluorescein succinimidyl ester (CFSE) (10 μM). WT BMDCs were incubated with 1Z105 (2 |M) and MPLA (1 μg/ml) overnight, and OVA (10 μg/ml) was added to the culture for the last 4 h of incubation. The cells were washed and cultured with CFSE labeled CD8⁺ OT-1 T cells for 3 days. OT-1 T cell proliferation was monitored by CFSE dilution using a flow cytometer.

### In Vivo Immunization Study Using OVA and Influenza Virus rHA Antigens

C57BL/6 WT, Myd88^{-/-}, or TrifLps2/Lps2 mice were i.m. immunized with 20 μg OVA with 1Z105 (200 nmol/animal, equivalent to 89.4 μg/dose), 1V270 (10.8 μg/dose, equivalent to 10 nmol/animal), or a combination of 1Z105 (89.4 μg/dose) and 1V270 (10.8 μg/dose) in a total volume of 50 μl on days 0 and 7. Vehicle (10% DMSO in saline) and AddaVax (1:1 ratio with antigen in saline) were used as controls. Sera were collected on days 0, 7, 14, 21, 28, and 35. Mice were sacrificed on day 35, and the spleens were harvested. Approximately 2.5×10⁶/ml spleen cells was dispersed into round-bottom microtiter plates in triplicate in a total volume of 200 μl complete RPMI 1640 and restimulated with either 100 μg/ml OVA or medium alone. The cultures were then incubated at 37° C., 5% CO₂, and the supernatants were harvested after 72 h. The levels of gamma interferon (IFN-γ) in the culture supernatants were measured by ELISA (BD Bioscience, San Jose, Calif.) according to the manufacturer's instructions. In parallel, splenocytes were stimulated with 10 μg/ml OVA class I (OVA257-264) or class II (OVA323-339) peptide on anti-IFN-γ antibody-coated enzyme-linked immunospot (ELISpot) plates for 18 h. The results are reported as numbers of IFN-spot-forming cells (SFC) per million cells as described below.

BALB/c mice were i.m. immunized with rHA (5 μg per mouse for rPR/8, rCal/09, cH2/1, and cH6/1 HAs and 2 μg per mouse for rVN/04 HA) plus 1Z105 (89.4 μg/dose), 1V270 (10.8 μg/dose), or a combination of 1Z105 (89.4 μg/dose) and 1V270 (10.8 μg/dose) in 10% DMSO-PBS. AddaVax was used at a 1:1 ratio with antigen in PBS. Control groups received antigen in 10% DMSO-PBS or a combination of 1Z105 (89.4 μg/dose) and 1V270 (10.8 μg/dose) in 10% DMSO-PBS without antigen. Fluzone was used at 50 ng/HA per mouse, and all immunizations were delivered i.m. into the gastrocnemius muscle in a total volume of 50 μl. Splenocytes were stimulated for 20 h on anti-IFN-γ antibody-coated ELISpot plates with a pool of PR/8 peptides acquired from BEI Resources (Manassas, Va.) at a final concentration of 2 μg/ml in DMEM (NR-18973; the first 25 peptides were pooled for a stock concentration of 20 μg/μl per peptide in DMSO). IFN-γ spot-forming cells were detected with the IFN-γ ELISpot ALP kit (3321-2A; Mabtech, Cincinnati, Ohio). Antigen specific B cells from splenocytes were quantified 5 days after reexposure to antigen (5 μg of rPR/8HAin PBS only administered i.m.) by plating them on ELISpot plates coated with rPR/8 protein (Strep tag purified) for 20 h, and detection of secreted antibody was performed with an anti-mouse IgG secondary antibody conjugated to horseradish peroxidase (HRP).

### Measurement of Antigen-Specific Antibodies

Anti-OVA antibodies of the IgG subclasses IgG1 and IgG2c were measured by ELISA. Each ELISA plate contained a titration of a previously quantitated serum to generate a standard curve. The titer of this standard was calculated as the reciprocal of the highest dilution of serum that gave an absorbance reading that was double the background. Serum samples were tested at a 1:100 dilution and reported as U/ml based on comparison with the standard curve. Anti-influenza virus antibodies were measured by ELISA using purified virus or rCal/09 (Strep tag purified) as the substrate by standard methods. Endpoint titers were defined as the highest dilution of serum that resulted in a signal three times above the background level. Total IgG, IgG1, and IgG2a were detected. Hemagglutination-inhibiting (HAI) titers to the PR/8 strain were assayed using trypsin-heat-periodate-inactivated sera according to established WHO methods.

### Murine Influenza Virus Challenge

All challenge viruses were grown in 8- to 10-day-old embryonated eggs. Murine 50% lethal doses (mLD₅₀) were determined in 6- to 10-week-old female BALB/c mice. The challenge viruses included A/Puerto Rico/8/1934(H1N1) (10 mLD₅₀; 300 PFU), mouse-adapted (5 passages through mouse lungs before amplification in embryonated eggs) A/Netherlands/602/2009(H1N1) (5 mLD₅₀; 100 PFU), mouse-adapted B/Florida/04/2006 (25 mLD₅₀; 80,000 PFU), and a 6-plus-2 reassortant of the HA (a low-pathogenic form with the polybasic cleavage site removed) and neuraminidase (NA) from A/Vietnam/1203/2004(H5N1) in the PR/8 background (5 mLD₅₀; 110 PFU). Mice were anesthetized with ketamine/xylazine and infected intranasally in a volume of 50 μl of PBS.

### Histological Examination for Reactogenicity

BALB/c mice were injected with 1Z105 (89.4 μg/dose), 1V270 (10.8 μg/dose), or a combination of 1Z105 (89.4 μg/dose) and 1V270 (10.8 μg/dose) in the gastrocnemius muscles in a total volume of 50 μl in the absence of antigen; 10% DMSO and 50% AddaVax in saline were used as controls. Twenty-four hours after injection, muscle tissues at the injection sites and sera were harvested. The muscles were fixed in 10% buffered formalin and embedded in paraffin. Sections 5 μm thick were stained with hematoxylin and eosin (H&E) and examined under a microscope.

### Quantitative RT-PCR

Harvested tissues were immediately frozen in liquid nitrogen and stored at −80° C. Total RNA was extracted from tissues or cells using an RNeasy minikit (Qiagen). cDNA synthesis was performed using iScript (Bio-Rad), and real-time (RT) PCRs were performed on the Bio-Rad iCycler IQ. The comparative Ct method was used to assess fold changes in expression of RNA transcripts between control and drug-treated mice. CT values were determined by subtracting the average glyceraldehyde-3-phosphate dehydrogenase (GAPDH) RNA gene threshold cycle (CT) values from each test CT value. CT values were normalized by the control calibrator CT value. TaqMan Gene Expression Assays (murine IL-6, keratinocyte chemoattractant [KC], monocyte chemotactic protein 1 [MCP-1], and MIP-1α) were purchased from Life Technologies.

### Statistical Analysis

For continuous outcomes, the data are represented as means and standard errors of the mean (SEM). A two-tailed Student t test or Mann-Whitney U test was used to compare two groups, and one-way analysis of variance (ANOVA) with Dunnett's post hoc test was used to compare multiple groups with the control. The ANOVA model assumption of equal variances across groups was checked using Bartlett's test of homogeneity of variances. For influenza virus serum HAI and endpoint titer data, the Kruskal-Wallis test with Dunn's correction with multiple comparisons was used to assess significance compared to the no-adjuvant control. When there was clear evidence of variance heterogeneity, nonparametric multiple-contrast tests were used to compare an adjuvant group to the controls with Dunnett's adjustment for multiple comparisons using the R-nparcomp package. For weight loss curves, t tests correcting for multiple comparisons by the Holm-Sidak method were used to compare each day's weight to that of the no-adjuvant control, and no adjustment was made for performing comparisons on multiple days for the same endpoints. For survival curves, Kaplan-Meier curves were plotted, and the log rank test was performed to assess significance. Prism 6 (GraphPad Software) statistical software and R (version 3.1.0; [http://]www.r-project.org) were used to obtain P values for comparisons between groups (a P value of <0.05 was considered significant).

### 1Z105 Enhances Dendritic Cell Maturation and Antigen Presentation

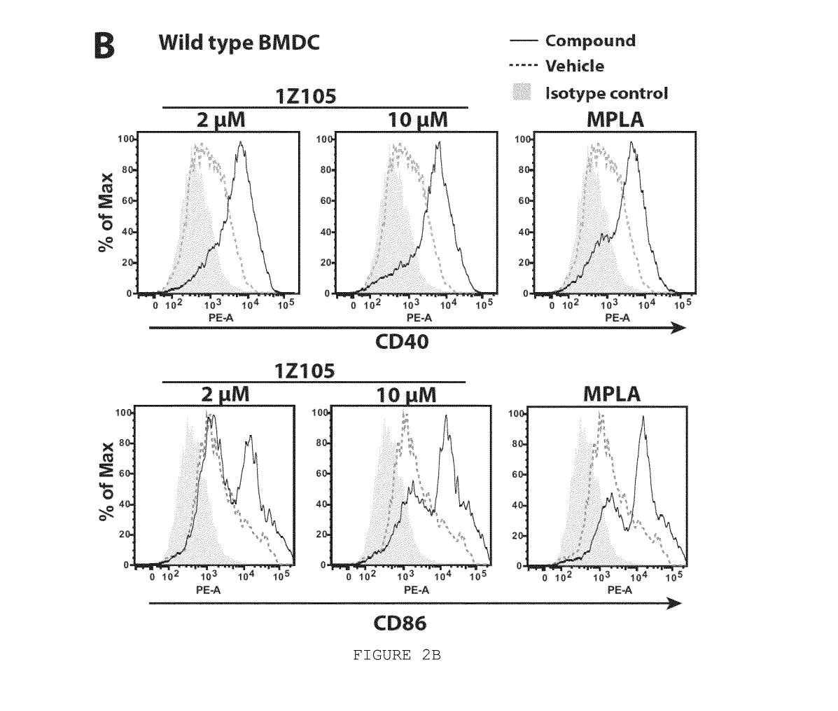

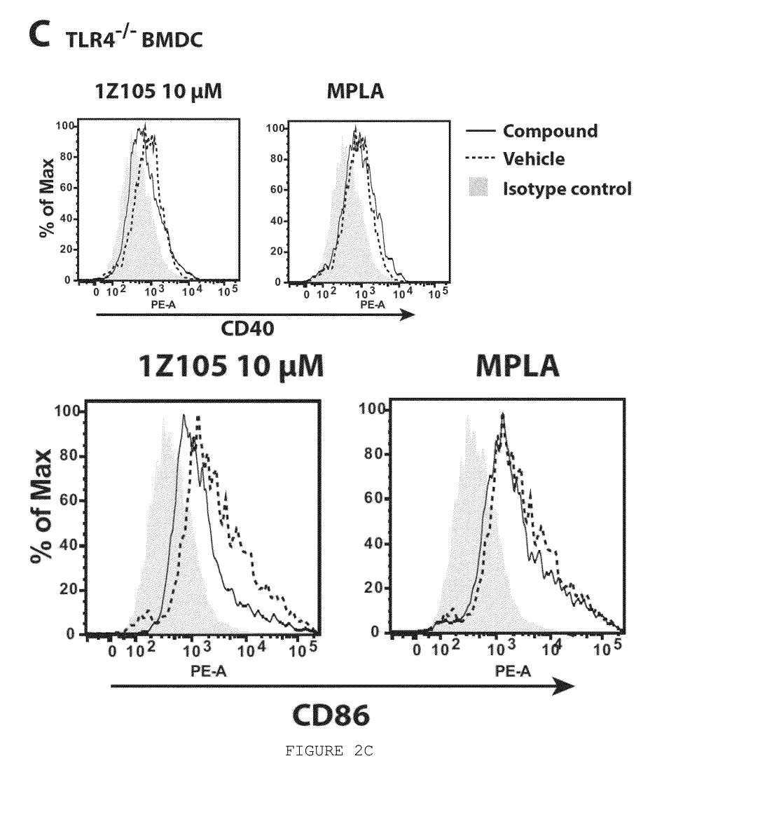

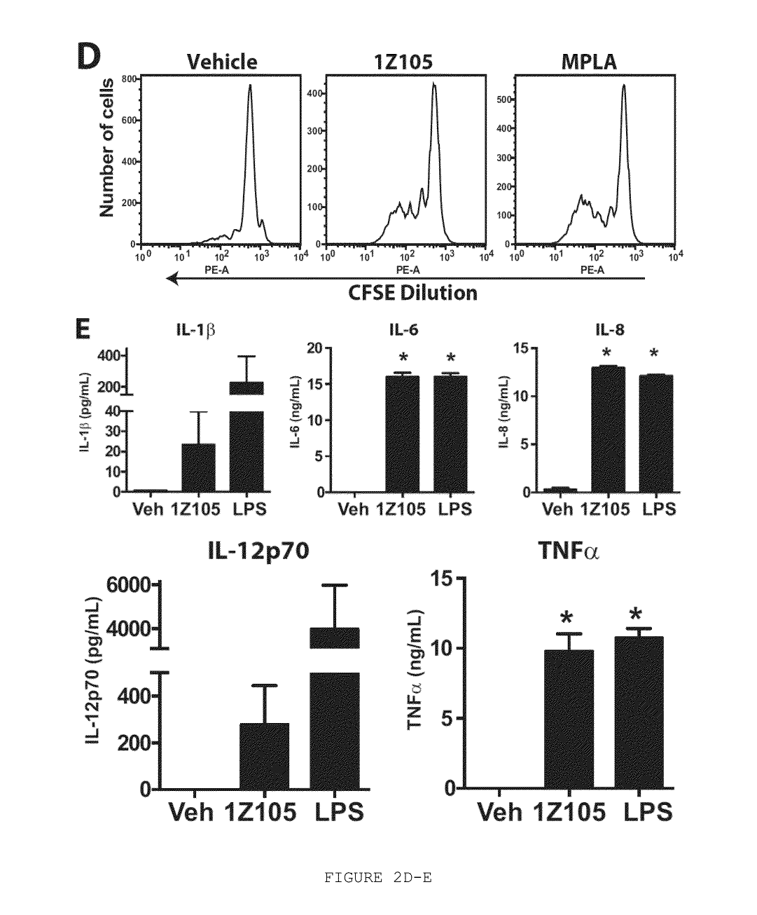

Adjuvants are added to vaccine antigens to potentiate antigen-specific immune responses and induce desired protective responses. Hence, key functions of adjuvants are directed at local immature DCs, inducing them to capture antigen, mature, and migrate to draining lymph nodes, where they prime naive T cells. Here, the adjuvant activity of a TLR4 agonist, 1Z105 (FIG. 1) was evaluated, with three in vitro assays to evaluate (i) antigen uptake, (ii) DC maturation, and (iii) cross-presentation to prime CD8+ T cells. BMDCs from C57BL/6 mice were compared after overnight exposure to 1Z105; MPLA, which is a semisynthetic TLR4 ligand; or vehicle for antigen uptake as assessed by incubation with fluorescently labeled OVA (FIG. 2A). After incubation with 1Z105 or MPLA at 37° C., an increased number of OVA-associated CD11chi BMDCs were observed, but no effect was observed with BMDCs incubated at 4° C. (FIG. 2A). Next, to evaluate whether 1Z105 enhanced DC maturation, the induction of costimulatory molecules (CD40 and CD86) on BMDCs incubated with 1Z105, MPLA, or vehicle was determined by flow cytometry. 1Z105 increased the surface expression of CD40 and CD86 at levels similar to that of MPLA (FIG. 2B). This was not observed in TLR4-deficient BMDCs (FIG. 2C). Lastly, antigen cross-presentation was tested by assaying the proliferation of OVA-specific CD8+ T cells (OT-1) incubated with BMDCs and OVA protein in the presence of 1Z105, MPLA, or vehicle. BMDCs incubated with 1Z105 or MPLA induced proliferation of OT-1 cells, indicating that they efficiently processed the extracellular OVA protein via the major histocompatibility complex (MHC) class I pathway (FIG. 2D). Furthermore, 1Z105 activated human monocyte-derived DCs, resulting in proinflammatory cytokine release (FIG. 2E). These in vitro evaluations strongly suggested that 1Z105 enhanced the antigen-presenting functions of DCs.

### Combination of 1Z105 with a TLR7 Ligand, 1V270, Induces Both Th1- and Th2-Associated Humoral Responses and Antigen Specific Cellular Immune Responses

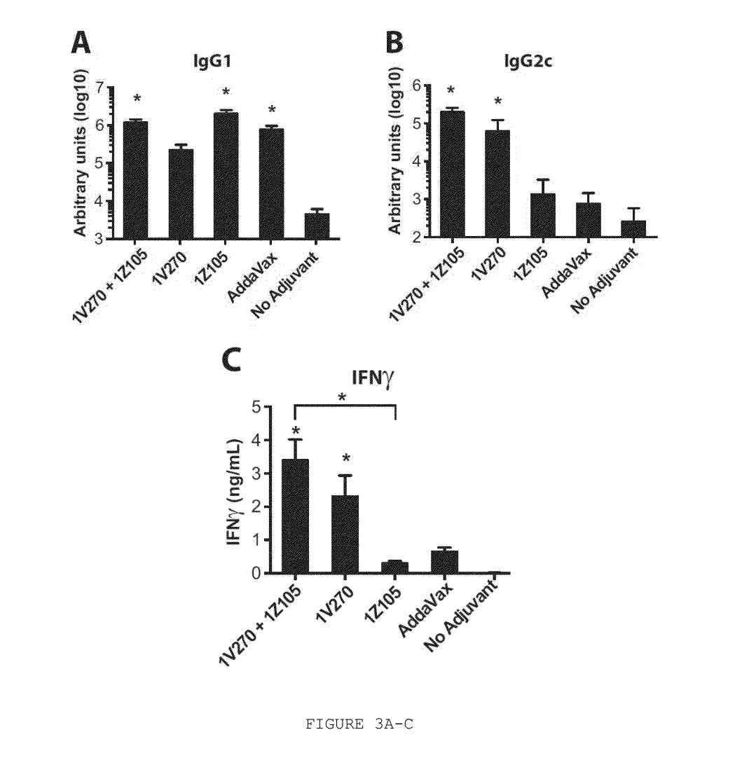

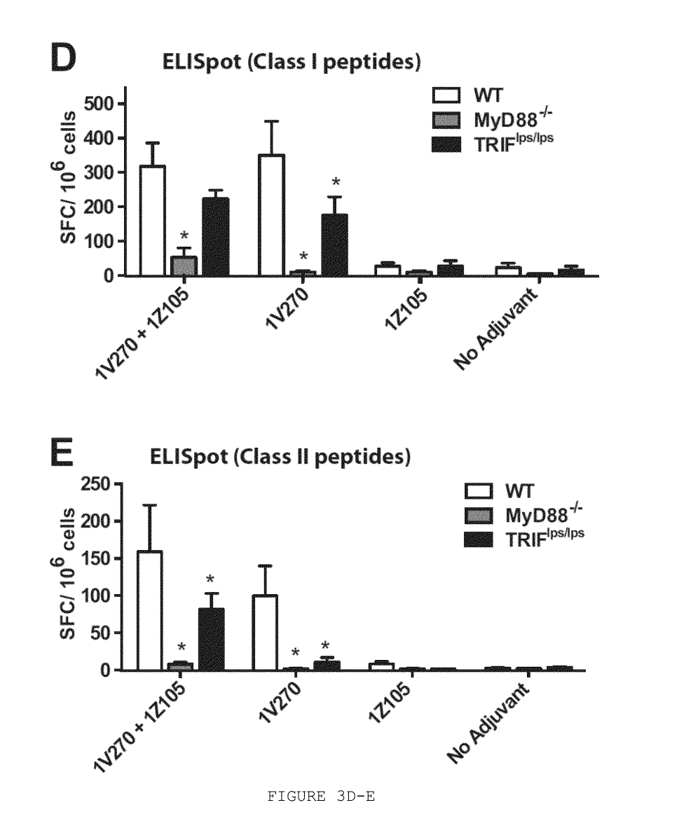

To develop an adjuvant that can be easily prepared and stably replicated in composition, synthetic molecules are preferable to natural products. Hence, a synthetic TLR7 ligand conjugated to a phospholipid, 1V270 (FIG. 1), previously shown to have protective effects in a murine infectious model was selected for study. C57BL/6 mice were immunized i.m. with a model antigen, OVA (20 μg/animal), plus 1Z105 (89.4 μg/dose), 1V270 (10.8 μg/dose), or a combination of 1Z105 and 1V270. AddaVax and unadjuvanted OVA in vehicle (no adjuvant) were used as controls. Immunoglobulin levels on day 35 in the sera indicated that 1Z105 induced significantly higher IgG1 (FIG. 3A), but not IgG2c (FIG. 3B), antibody titers than OVA alone, so that the antibody profile was similar to that of AddaVax. In contrast, 1V270 induced significantly higher IgG2c titers, but it did not induce IgG1 comparably to 1Z105. When the two ligands were combined, significant induction of both IgG1 and IgG2c was observed.

Furthermore, 1V270 and the combination of 1V270 and 1Z105 promoted OVA-specific IFN-γ release by T cells restimulated ex vivo (FIG. 3C). OVA-specific ELISpot assays demonstrated that splenocytes from mice immunized with antigen plus 1V270, or a combination of 1Z105 and 1V270, contained increased frequencies of IFN-γ-releasing T cells specific for OVA class I (FIG. 3D) and class II (FIG. 3E) peptides. These increased antigen-specific T cell frequencies were partially dependent upon the MyD88 and TRIF pathways (FIGS. 3D and E). These data indicate that MyD88 signaling is required for the adjuvant activity of 1V270 alone and in combination with 1Z105. TLR7 signaling utilizes MyD88, and the partial requirement for TRIF suggests that it may affect MyD88 signaling in antigen-presenting cells (APCs). TRIF is required for the TLR4-mediated expansion of T cells and may contribute to T cell development when 1V270 and 1Z105 are used in combination. Collectively, these findings indicated that the combination of 1Z105 and 1V270 could induce robust Th1- and Th2-associated humoral responses, in addition to MHC class I- and II-restricted T cell responses.

### 1Z105 and 1V270 with a Recombinant Hemagglutinin Antigen Induce Rapid Protective Immunity to a Homologous Strain of Influenza A Virus after a Single Immunization

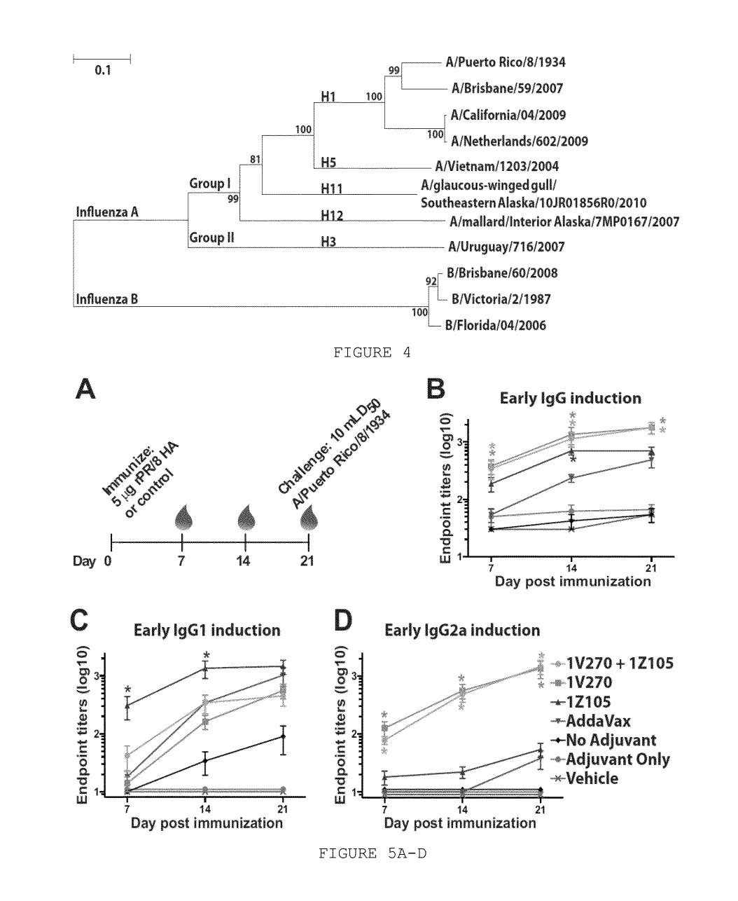

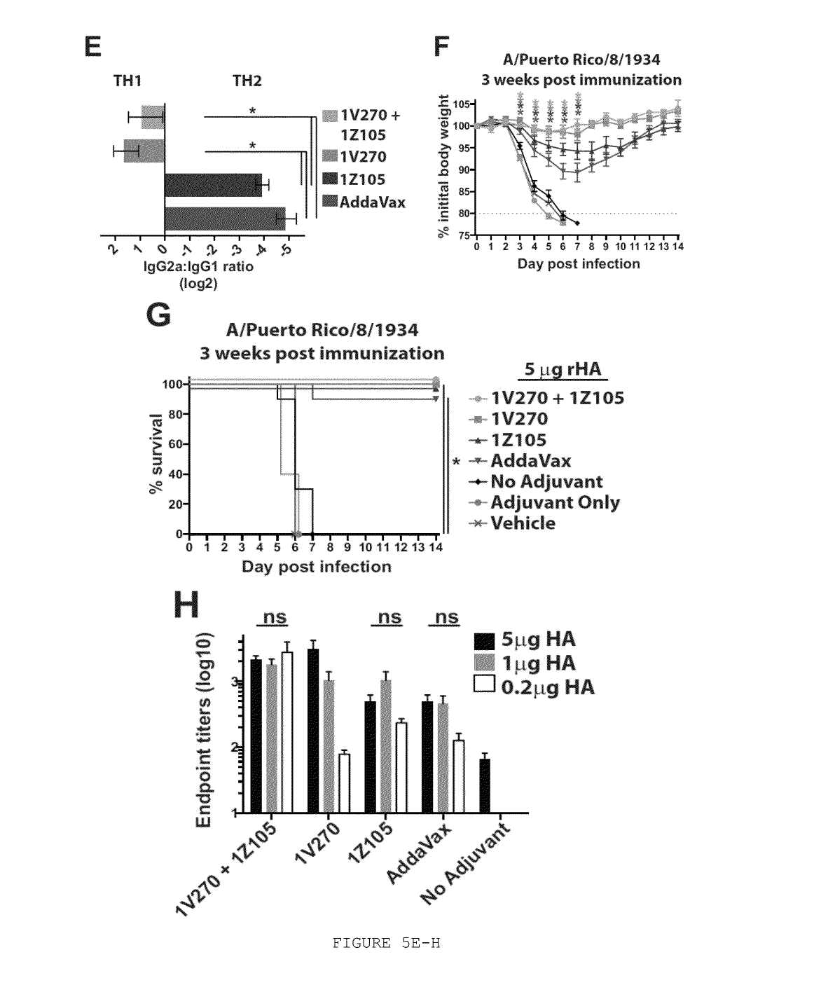

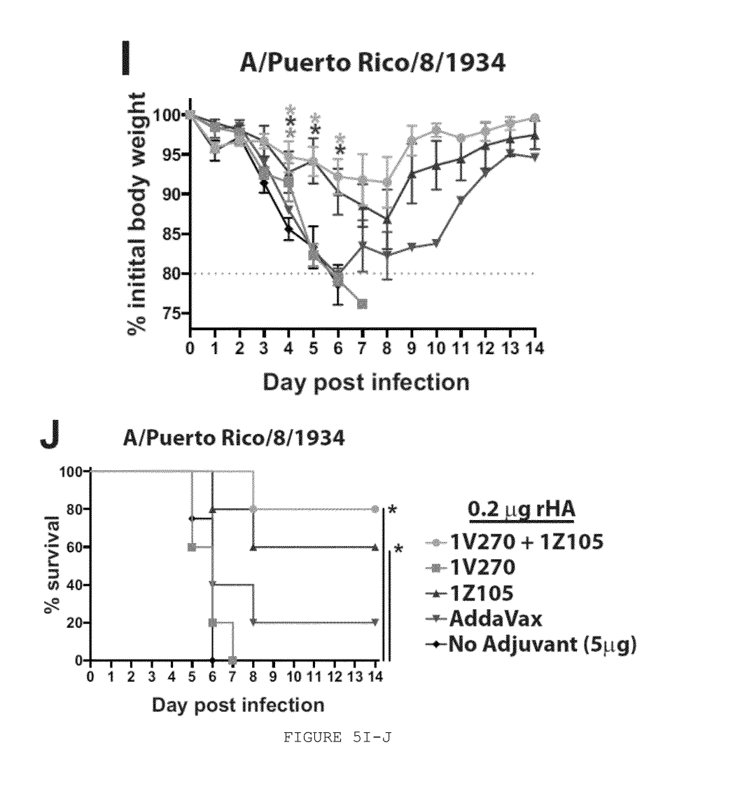

In order to assess the efficacy of 1Z105 and 1V270 as bona fide vaccine adjuvants, the mouse model for influenza virus vaccination and challenge was employed. Several HA antigens and challenge viruses were utilized to test various models of HA-directed immunity. The phylogenetic relationships of the HAs relevant to this work are depicted in FIG. 4. Current vaccines rely upon the induction of strain-specific neutralizing antibodies directed toward the antigenic regions of the influenza virus HA. Therefore, rHA derived from A/Puerto Rico/8/1934 (PR/8) was chosen as the vaccine antigen in order to concentrate upon HA-specific immunity that is protective against a homologous challenge. BALB/c mice were immunized with 5 μg of rPR/8 HA on day 0 and bled on days 7, 14, and 21, as schematized in FIG. 5A. The early seroresponse was assessed for the induction of antigen-specific total IgG (FIG. 5B), IgG1 (FIG. 5C), and IgG2a (FIG. 5D) as early as 7 days after a single immunization. Both 1Z105 and 1V270, in addition to AddaVax, which was included as a reference, induced rapid seroconversion to the rPR/8 HA. 1Z105 and AddaVax produced predominately IgG1 in a Th2-type response, as demonstrated by the IgG2a/IgG1 ratio (FIG. 5E). 1V270, alone and in combination with 1Z105, induced both IgG1 and IgG2a in a more balanced Th1-Th2-type response (FIG. 5E). The control groups included mice receiving rPR/8 HA without adjuvant (no adjuvant) or 1V270 and 1Z105 without antigen (adjuvant only) and animals receiving the vehicle.

Three weeks after immunization with 5 μg of rPR/8 HA, all groups were challenged with 10 mLD50 of PR/8 virus. Vaccination with HA adjuvanted by 1Z105 and 1V270, alone or in combination, provided protective efficacy against morbidity, as measured by weight loss (FIG. 5F), and mortality (FIG. 5G) in response to the viral challenge. 1V270, alone or in combination with 1Z105, also restricted weight loss compared to AddaVax. The adjuvant-only control group demonstrated morbidity and mortality similar to those vaccinated with vehicle, confirming that the protection afforded by 1V270 and 1Z105 is mediated by an enhanced response to the antigen rather than the nonspecific induction of antiviral immunity.

In addition to enhancing the immune response and inducing a bias in the T helper response, adjuvants are known to reduce the amount of antigen needed to induce a protective response. 1Z105 and 1V270 were assayed for their antigen-sparing properties by immunizing mice with 5, 1, or 0.2 μg of rPR/8 HA. In FIG. 5H, the total serum IgG endpoint titers were assayed by ELISA 3 weeks after immunization. For the combination of 1V270 and 1Z105, 1Z105 alone, and AddaVax, no significant difference in serum IgG titers was detected among the 3 rHA doses, indicating robust antigen-sparing properties. Mice receiving 0.2 μg of rHA were challenged 3 weeks postimmunization with 10 mLD50 of PR/8 virus and followed for morbidity (FIG. 5I) and mortality (FIG. 5J). 1Z105, alone or combined with 1V270, significantly minimized morbidity and mortality compared to the no-adjuvant control group receiving 5 μg of rHA. In terms of antigen sparing, the combination of 1V270 and 1Z105 conferred an advantage over using either adjuvant alone when endpoint titers (FIG. 5H), morbidity (FIG. 5I), and mortality (FIG. 5J) were compared. In summary, the data presented in FIG. 5 suggested that 1Z105 and 1V270 had efficacies comparable to or better than that of AddaVax as influenza virus vaccine adjuvants and warranted characterization with more contemporary antigens.

### 1Z105 and 1V270 Induce Rapid Protective Immunity to the Pandemic H1N1 Virus and an Avian H5N1 Subtype Virus and Enhance the Immunogenicity of Fluzone

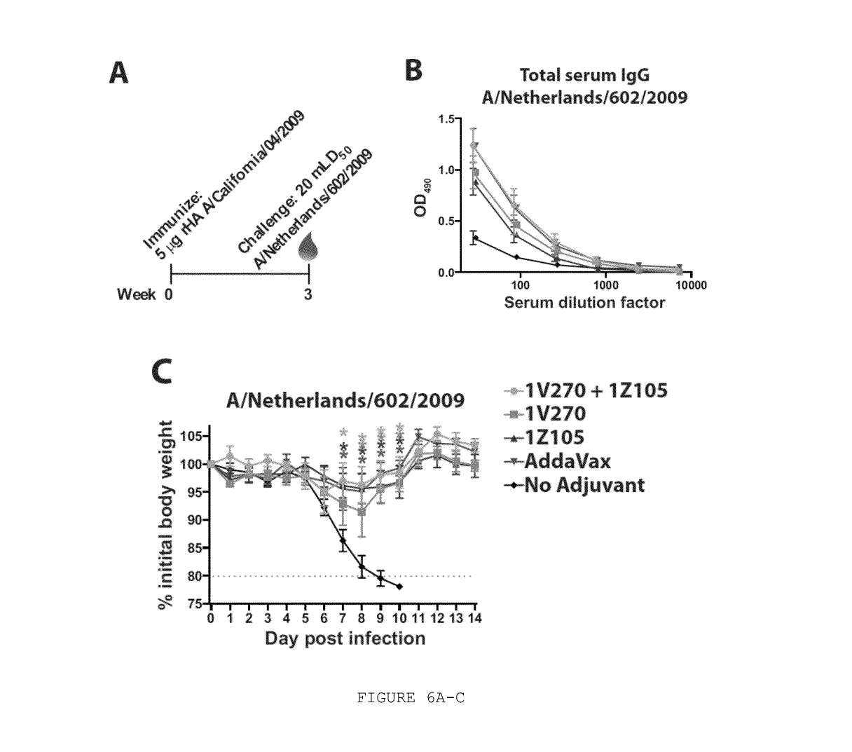

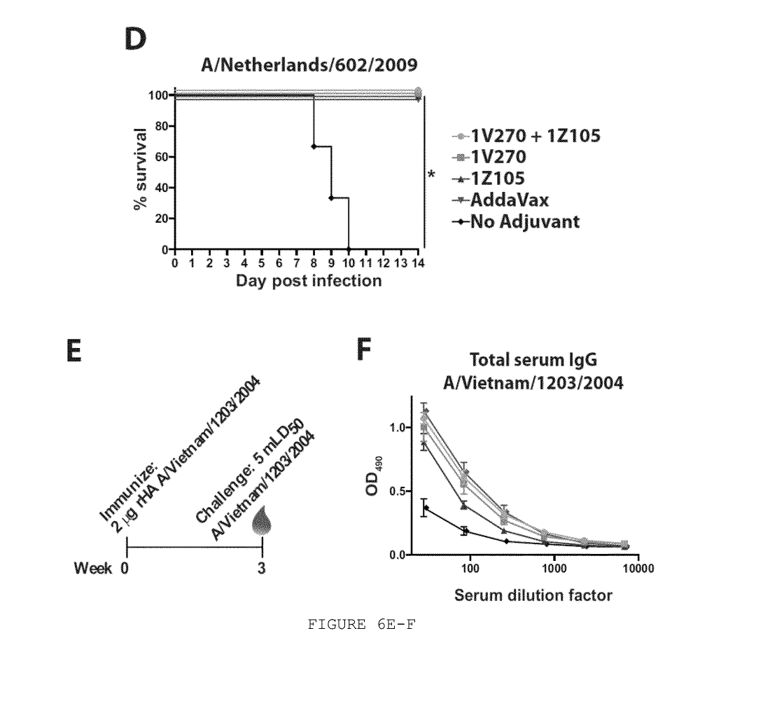

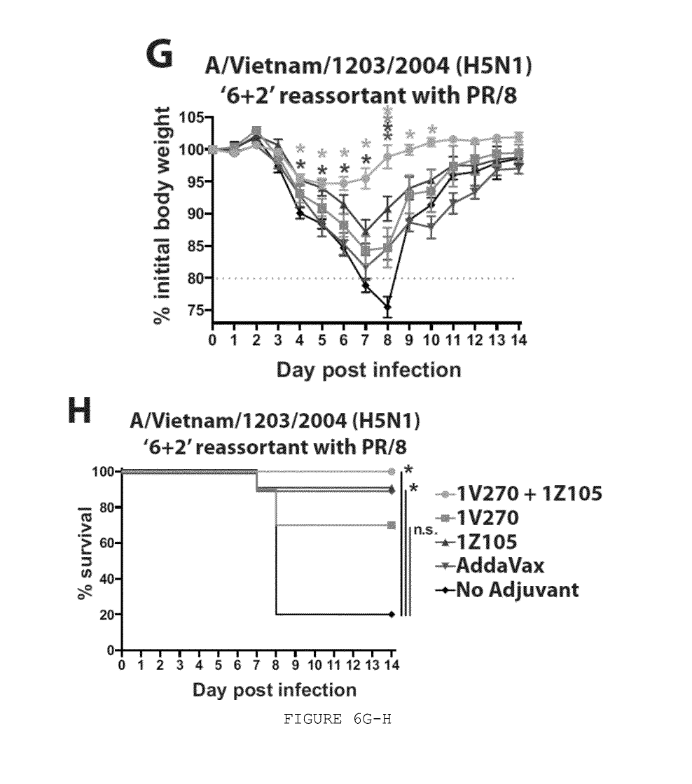

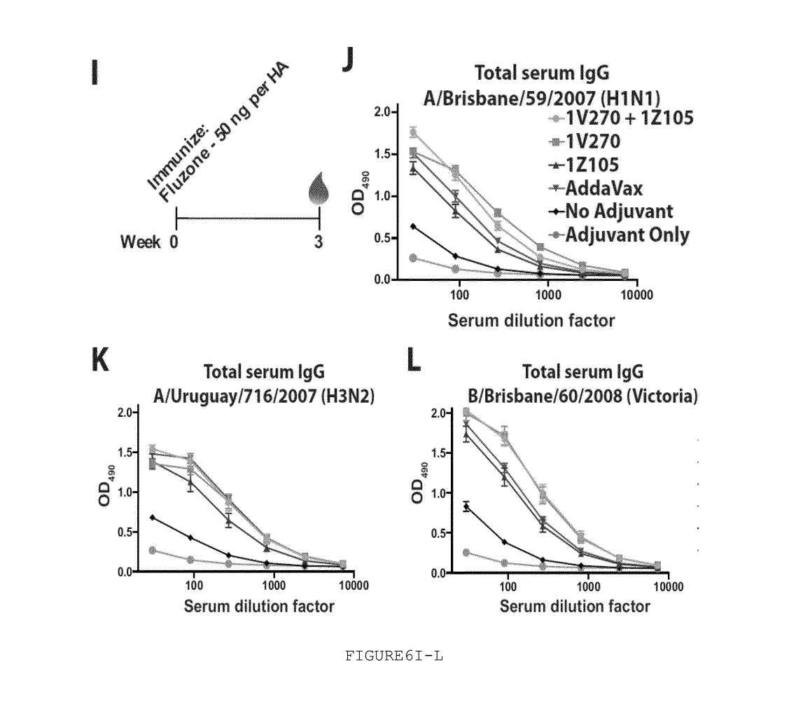

After assessing the efficacy of 1Z105 and 1V270 with rPR/8 HA, antigens more relevant to the development of present-day influenza virus vaccines were tested with the TLR ligands. rHA derived from Cal/09 was used to determine the efficacy of 1Z105 and 1V270 for the induction of protective immunity to the 2009 pandemic H1N1 (pH1N1) virus (FIG. 6A). 1Z105 and 1V270, alone and in combination, and AddaVax enhanced the IgG seroresponse (FIG. 6B) and served to restrict morbidity, as measured by weight loss (FIG. 6C), and mortality (FIG. 6D) after a lethal challenge with a mouse-adapted pH1N1 virus, A/Netherlands/602/2009, which is essentially homologous to Cal/09 (FIG. 4).

Avian-origin influenza A viruses of the H5N1 subtype are of concern for their potential to cause a human pandemic with a high mortality rate, and efforts to develop vaccines against H5 subtype avian influenza viruses have demonstrated that H5 subtype HAs are poorly immunogenic and may require the use of an adjuvant. Mice were immunized with 2 μg of rHA derived from A/Vietnam/1203/2004 (VN/04), a highly pathogenic avian H5N1 subtype virus, plus adjuvant or in vehicle alone (FIG. 6E). 1Z105, 1V270, and AddaVax enhanced the IgG seroresponse (FIG. 6F) when administered with rHA. 1Z105, alone or combined with 1V270, significantly minimized morbidity (FIG. 6G) and mortality (FIG. 6H) induced by 5 mLD50 of a 6-plus-2 reassortant virus consisting of the VN/04HAandNAin the PR/8 background. Notably, the combination of 1Z105 and 1V270 minimized morbidity, as assayed by weight loss, more effectively than either adjuvant alone and provided 100% survival after challenge.

The vast majority of influenza virus vaccines in present-day use consist of split virions grown in embryonated chicken eggs. These vaccines are time-consuming to produce and depend upon the availability of eggs. The use of adjuvants with split vaccines can enhance their immunogenicity and reduce the amount of antigen required to generate a seroresponse (20, 46). 1Z105 and 1V270 were assayed for the ability to enhance the seroresponse to the 2009-2010 seasonal Fluzone, a trivalent vaccine consisting of three viral components: A/Brisbane/59/2007 (H1N1), A/Uruguay/716/2007 (H3N2), and B/Brisbane/60/2008 (Victoria lineage). Mice were immunized with Fluzone and bled 3 weeks later (FIG. 6I). 1Z105 and 1V270, alone and in combination, and AddaVax enhanced the seroresponse to all three viral components, as assayed by ELISA (FIG. 6J to L). These data confirmed that 1V270 and 1Z105 rapidly induce a protective humoral response comparable to or better than AddaVax when administered with relevant influenza virus antigens, and this indicated a need to characterize the long-term effects of the adjuvant.

### 1Z105 and 1V270 Induce Sustained Protective Immunity to Influenza A Virus after a Single Immunization with rHA

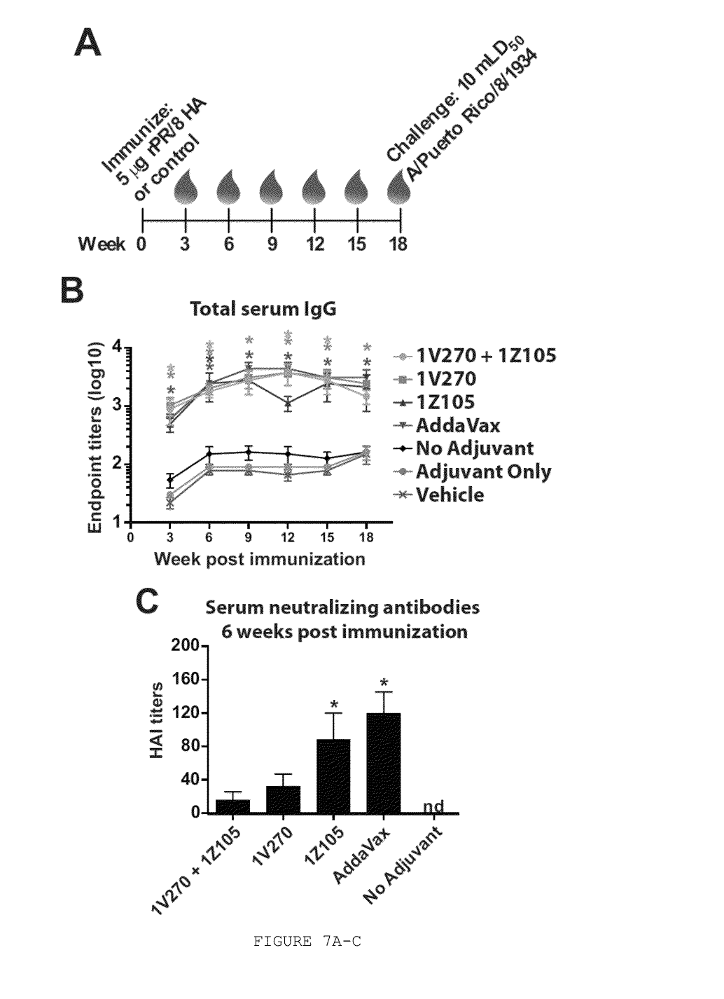

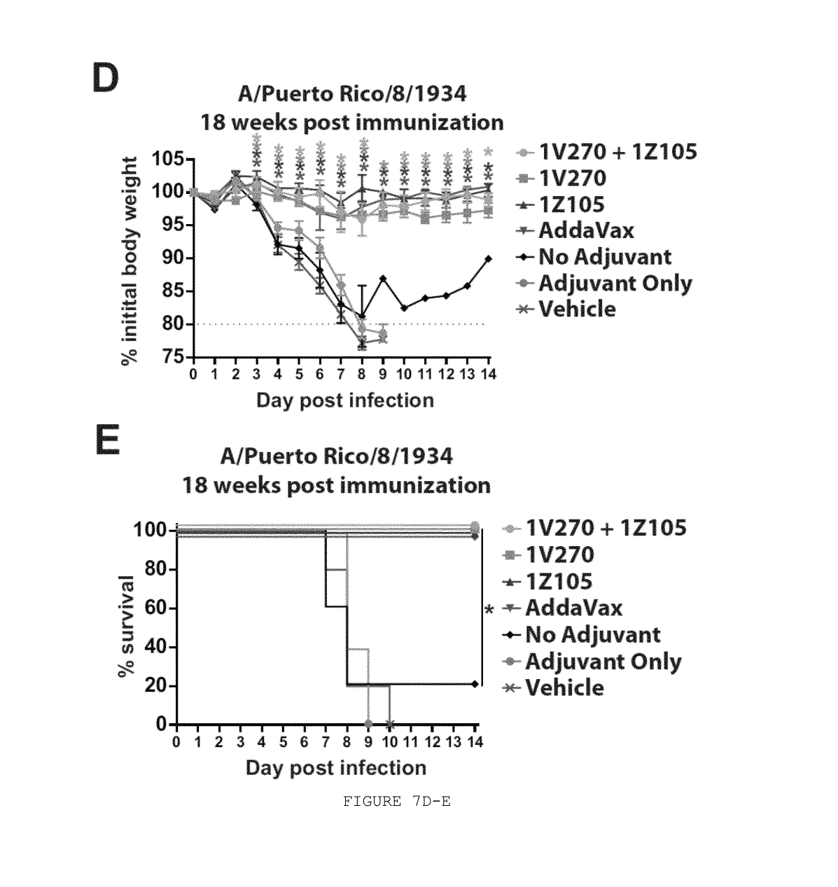

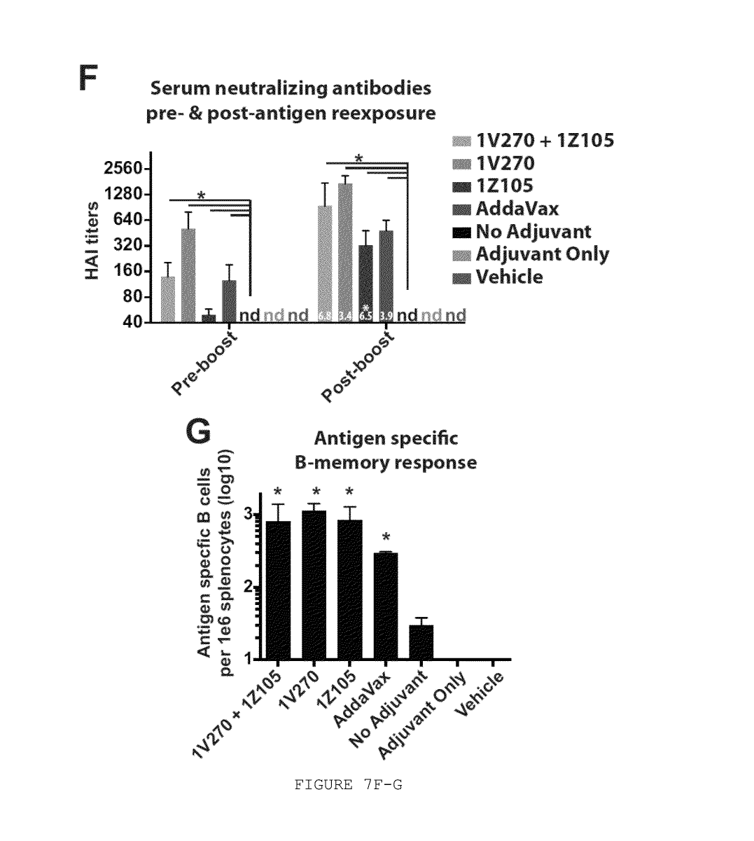

After characterizing the rapid response to rPR/8 HA induced by 1Z105 and 1V270, the long-term response to antigen was assessed. Mice were immunized with 5 μg of rPR/8HAand bled every 3 weeks up to 18 weeks postimmunization, at which point they were challenged to determine whether protective immunity was sustained (FIG. 7A). The total serum IgG reactive to antigen was measured by ELISA (FIG. 7B). 1Z105 and 1V270, alone and in combination, and AddaVax induced a robust and sustained IgG response. Sera were assayed for neutralizing capacity by their ability to inhibit PR/8 virus from hemagglutinating chicken red blood cells. HAI titers were detectable by 6 weeks postimmunization, as the serum endpoint titers continued to rise (FIGS. 7B and C). After 18 weeks, mice were challenged with 10 mLD50 of PR/8 virus and followed for morbidity (FIG. 7D) and mortality (FIG. 7E). 1V270 and 1Z105 significantly reduced weight loss and provided 100% survival after the lethal challenge, indicating that antigen-specific antiviral immunity induced by the adjuvants is long lived.

To gain a better appreciation of the quality of the long-lived immune response, mice were immunized one time with 5 |g of rPR/8 HA with or without adjuvant and assayed for memory response upon reexposure to antigen. In one cohort (FIGS. 7F and G), all groups of mice (including those originally receiving adjuvant only or vehicle) were i.m. administered 5 μg of rPR/8 HA in PBS only more than 6 months after the primary immunization. The mice were bled prior to and 5 days after antigen reexposure and assayed for serum HAI titers to PR/8 virus (FIG. 7F). All of the adjuvants induced sustained HAI titers after the primary immunization that were significantly greater than those of the no-adjuvant control group (FIG. 7F, left). Five days after reexposure to antigen, serum HAI titers in the adjuvanted groups were significantly elevated compared to the unadjuvanted control (FIG. 7F, right). The fold increase in HAI titer induced by the boost (indicated on the bars in FIG. 7F, right) was significant only in the 1Z105 group at 5 days after immunization; however, significant expansion of antigen-specific B cells from splenocyte preparations was readily detectable by ELISpot in all of the adjuvanted groups (FIG. 7G). These antigen-specific B cells do not represent a primary response to the antigen exposure, as they were undetectable in the groups that received adjuvant only and vehicle at the time of the primary immunization.

In a second cohort, mice were immunized with 5 μg of rPR/8 HA with or without adjuvant, and splenocytes were isolated 6 months after the immunization, stimulated overnight with a peptide pool derived from PR/8 HA, and assayed for IFN-γ-producing T cells by ELISpot (FIG. 7H). Similar to the prior observations in studies using OVA, the Th1-type adjuvant 1V270, alone or combined with 1Z105, induced antigen-specific IFN-γ T cells that were long lived. These data confirmed that 1V270 and 1Z105 are comparable to or better than AddaVax by several measures, and this prompted work to evaluate the efficacy of 1V270 and 1Z105 as adjuvants in heterologous vaccination and challenge models.

### 1V270, Alone or in Combination with 1Z105, Induces Cross Protective Immunity to Heterologous Influenza Viruses

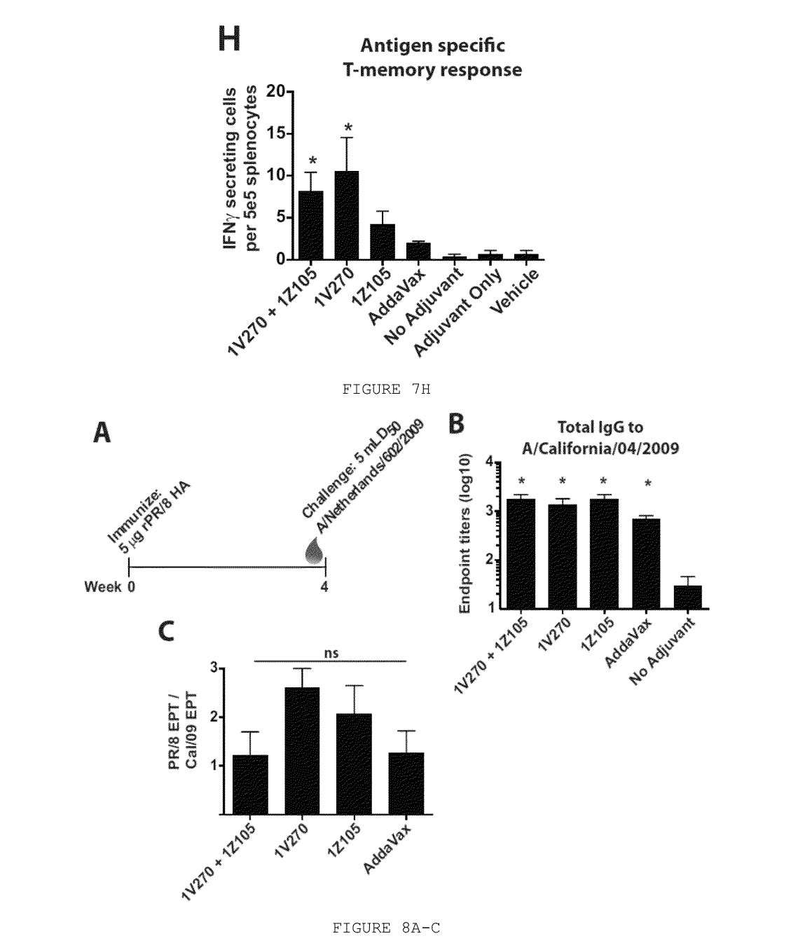

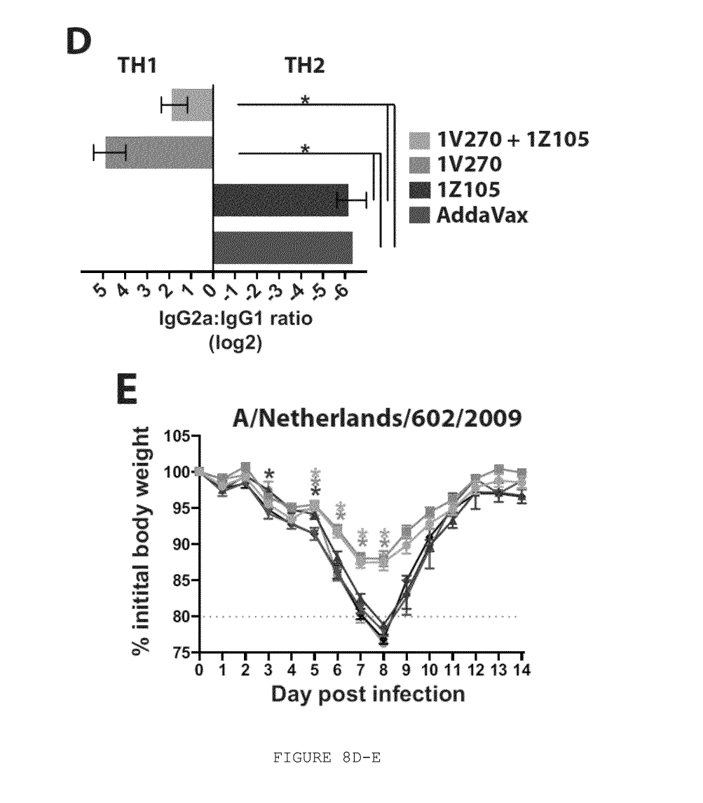

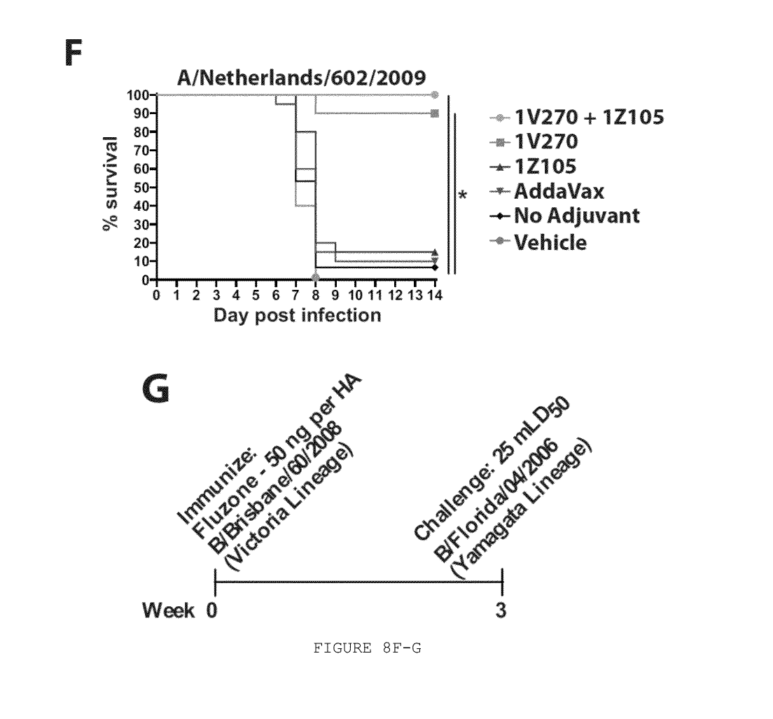

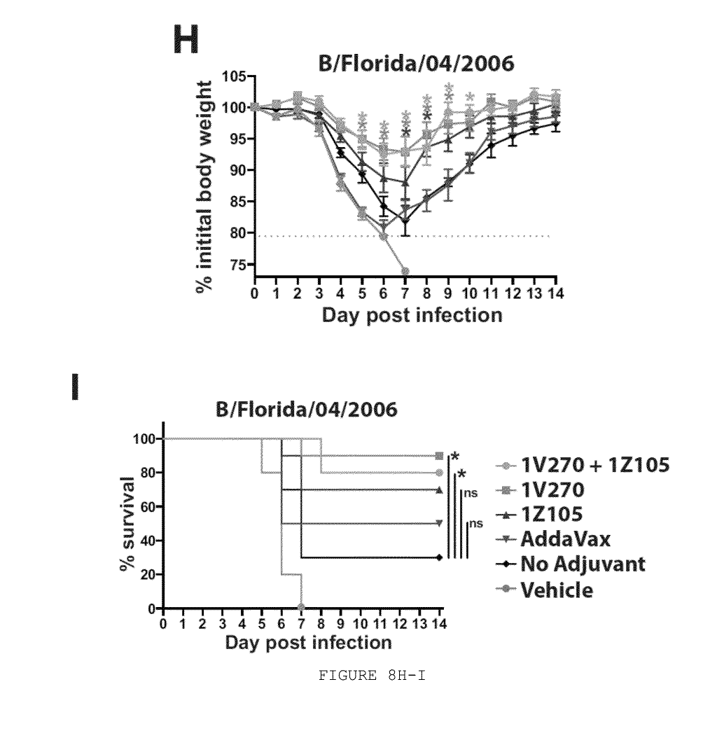

Some influenza virus vaccine adjuvants have been demonstrated to induce cross-protective responses, and cross-protection has been associated with the induction of Th1-type immune responses. Therefore, assays were performed to determine whether 1V270 and 1Z105 could induce heterologous protection from influenza viruses in two different assays. First, mice were immunized with 5 μg of rPR/8 HA with or without adjuvant and assayed for cross-reactive antibodies and cross-protective immunity to the pandemic 2009 H1N1 virus (FIGS. 4 and 8A). 1V270 and 1Z105, alone and in combination, as well as AddaVax, induced significantly higher heteroreactive serum IgG titers than unadjuvanted protein alone (FIG. 8B). A significant difference was not observed in the ability of any adjuvant to induce more cross-reactive total IgG to the pandemic HA relative to the total IgG induced to the PR/8 immunogen (FIG. 8C). The subtypes of these cross-reactive antibodies were similar to those discussed above, with adjuvants containing 1V270 inducing more IgG2a while 1Z105 and AddaVax produced predominately IgG1 (FIG. 8D). Animals were subsequently challenged with 5 mLD₅₀ of a mouse-adapted pandemic H1N1 virus (A/Netherlands/602/2009) and followed for morbidity, as measured by weight loss (FIG. 8E), and mortality (FIG. 8F) induced by the heterologous viral challenge. 1V270, alone and combined with 1Z105, significantly reduced weight loss and mortality resulting from the viral challenge. As single agents, the Th2-type adjuvants 1Z105 and AddaVax did not induce protective immunity in this heterologous-challenge model. The cross protective efficacy of the adjuvants correlates with the induction of a Th1-type response (FIG. 8D) rather than total cross-reactive serum IgG levels (FIG. 8C).

Influenza B viruses are unique among human influenza viruses in that two closely related strains from two distinct lineages, Victoria and Yamagata, cocirculate. Despite a high degree of homology between B virus HAs from different lineages (FIG. 4), the vaccines are not necessarily cross-protective. Mice were immunized once with the 2009-2010 seasonal Fluzone, which contains B/Brisbane/60/2008 (Victoria lineage) (FIG. 8G). Three to 4 weeks after the immunization, mice were challenged with 25 mLD50 of a mouse-adapted heterologous influenza B virus (B/Florida/04/2006; Yamagata lineage) (FIG. 8G), and they were followed for morbidity, as measured by weight loss (FIG. 8H), and mortality (FIG. 8I). 1V270, alone and in combination with 1Z105, significantly reduced morbidity and mortality compared to the no-adjuvant group. 1Z105 also reduced morbidity by restricting weight loss on days 7 and 8 post infection. These data indicated that 1V270 and the combined 1Z105 and 1V270 adjuvant induce cross-protective immunity. Therefore, their adjuvant activities using candidate universal vaccine constructs were evaluated.

### 1V270, Alone or Combined with 1Z105, Induces Protective Heterosubtypic Immunity to the Conserved HA Stalk Domain when Administered with Chimeric Hemagglutinins

In addition to the challenge of addressing antigenic drift within an HA subtype, influenza A viruses sporadically reassort to produce antigenically novel viruses capable of causing serious pandemic outbreaks in a human population with little preexisting immunity. Therefore, the development of universal influenza virus vaccines based upon conserved epitopes is being fervently pursued. Among the most promising vaccine targets is the conserved HA stalk domain, and adjuvants have been demonstrated to play an essential role in the development of HA stalk directed immunity in a mouse model.

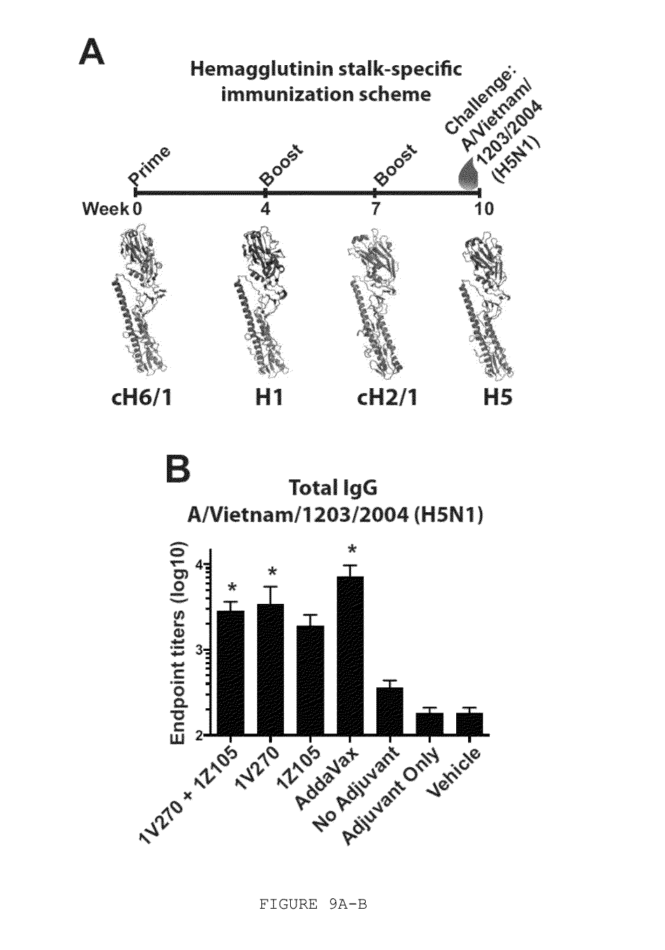

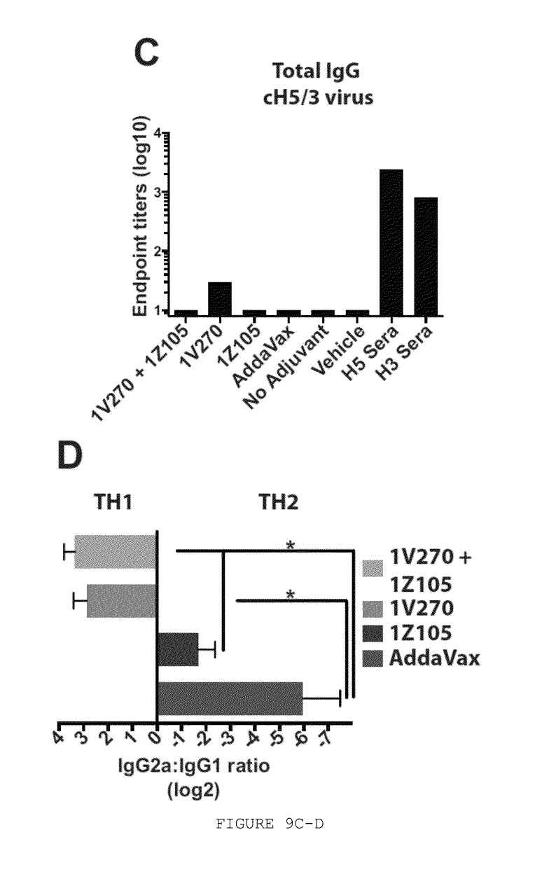

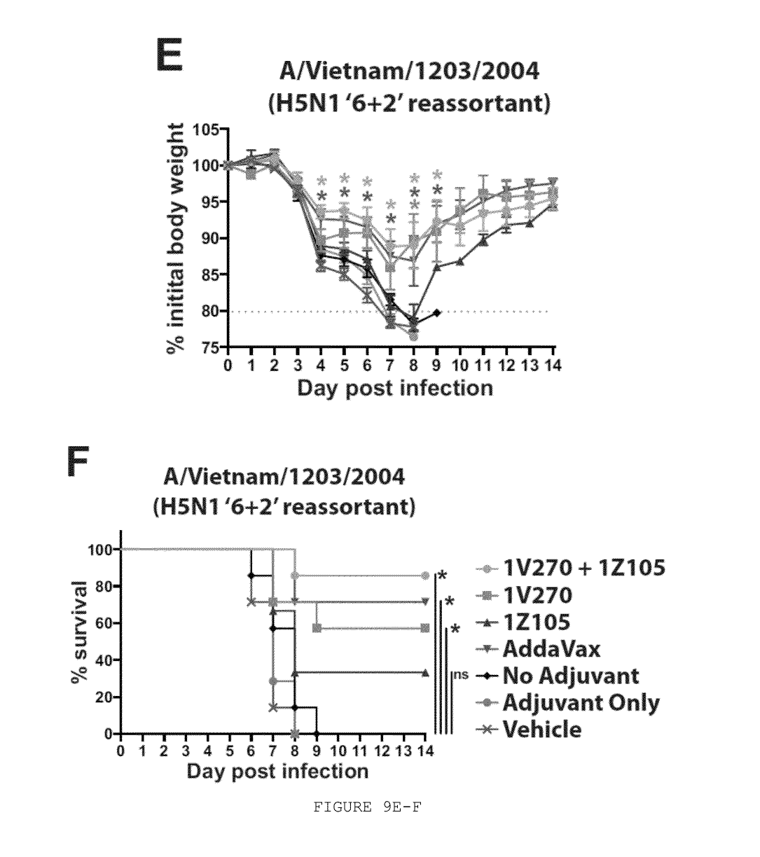

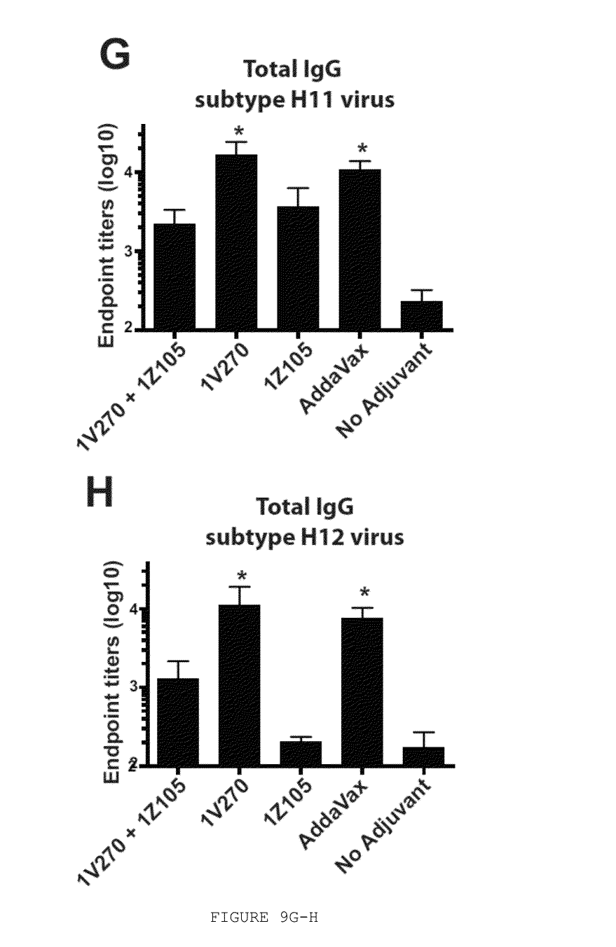

1Z105 and 1V270 were assayed for their abilities to induce HA stalk-directed immunity and to protect against a heterosubtypic viral challenge via sequential immunization with candidate universal vaccine constructs, cHAs (FIG. 9A). cHAs allow the antigenic separation of the globular head domain and the stalk domain by making novel combinations between the globular heads and stalks of different HA subtypes. In this immunization strategy, mice were repeatedly exposed to the rH1 subtype stalk (PR/8 strain), while the globular head domain was varied for each immunization by sequentially administering recombinant cH6/1, H1, and cH2/1 (FIG. 9A). Mice remained naive to the H5, H11, and H12 subtype globular heads.

Serum IgG levels with heterosubtypic reactivity to the VN/04 (H5) HA, based upon the conservation between group IHA stalks, were assayed by ELISA (FIGS. 4 and 9B). Compared to the no adjuvant control group, 1V270 and 1Z105, 1V270, and AddaVax induced significantly higher reactivity to the H5 subtype HA. To confirm that this reactivity is specific to the group I stalk and not the H5 subtype head, the sera were assayed by ELISA with cH5/3 (the H5 subtype globular head on the H3 subtype group II stalk) virus as the substrate (FIG. 9C). As expected, seroreactivity was ablated by replacing the group I stalk with the group II stalk. The subtype profiles were similar to those seen in other assays with 1V270, alone and in combination with 1Z105, inducing IgG2a while 1Z105 and AddaVax alone induced primarily IgG1 (FIG. 9D). Mice were challenged with 5 mLD50 of a 6-plus-2 reassortant virus consisting of the VN/04 HA and NA (H5N1) in the PR/8 background and followed for morbidity, as measured by weight loss (FIG. 9E), and mortality (FIG. 9F). 1V270, alone or combined with 1Z105, and AddaVax significantly restricted weight loss and mortality induced by the viral challenge. To further assay the breadth of HA stalk-based seroreactivity, more distant group I Has were assayed, including those of the H11 (FIG. 9G) and H12 (FIGS. 4 and 9H) subtypes. 1V270 and AddaVax induced IgG titers that reacted to H11 and H12 subtypes significantly better than the unadjuvanted control.

The accumulated data indicated that 1V270 and 1Z105, and their combination, induced rapid, potent, long-lasting, and cross protective immunity against influenza virus infection. Therefore, preclinical safety studies were initiated to assess the reactogenicity of 1V270 and 1Z105.

### Administration of the Combined 1Z105 and 1V270 Adjuvant Elicits Little Reactogenicity

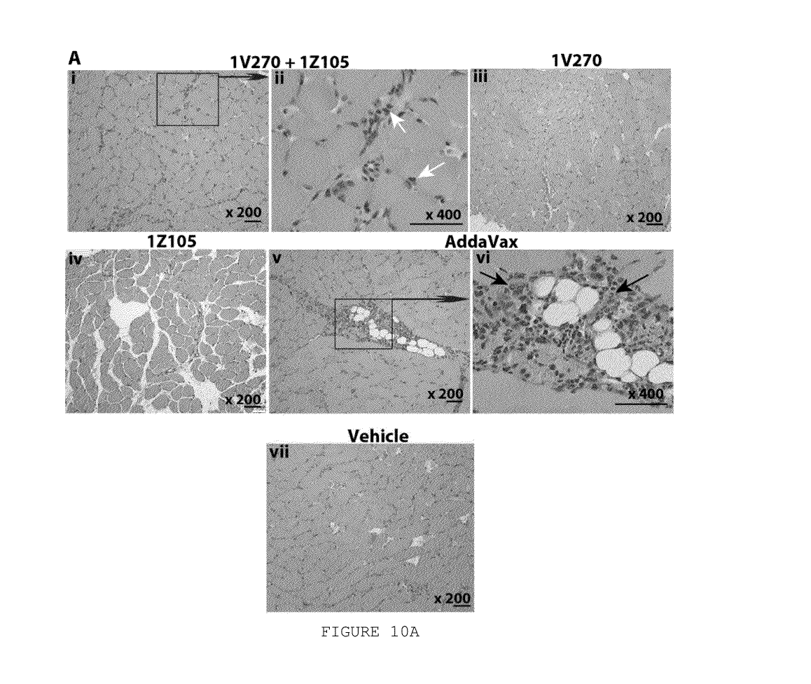

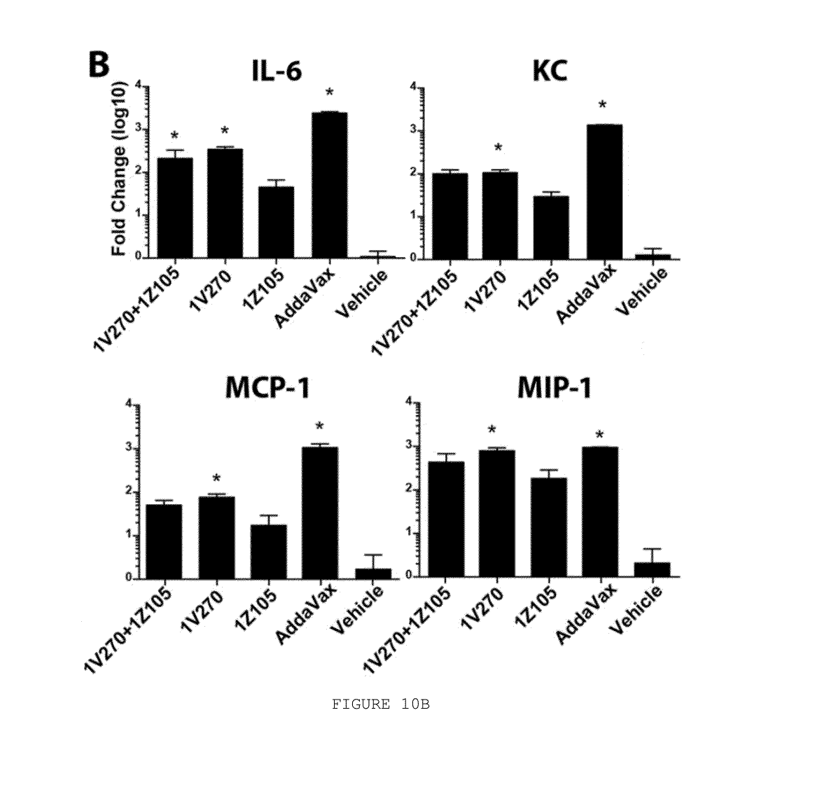

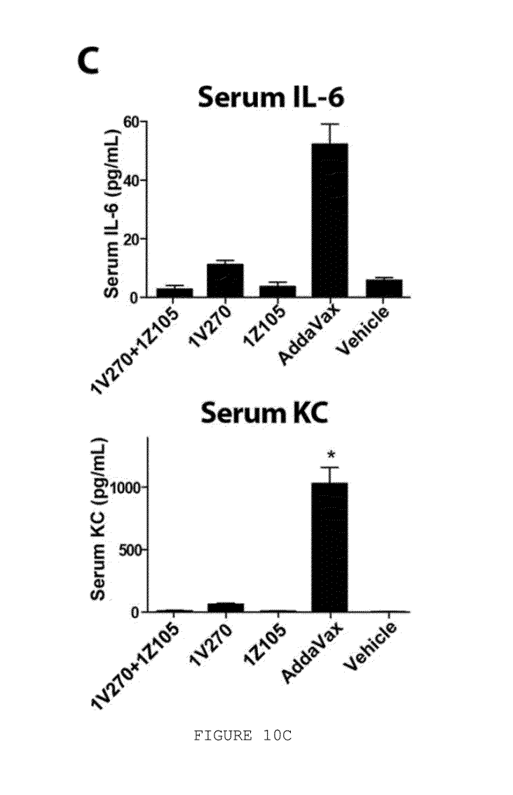

To evaluate the reactogenicity of the adjuvants, gastrocnemius muscles were harvested from BALB/c mice injected with the combination of 1Z105 and 1V270, 1V270, 1Z105, AddaVax, or vehicle alone. Histological examination of H&E-stained muscle sections showed minimal cellular infiltration, with mononuclear cells and a few polynuclear cells in the muscles injected with the combination of 1Z105 and 1V270 (FIG. 10A, i and ii) or 1V270 (iii) or 1Z105 (iv) alone. In contrast, markedly increased cellular infiltration, including both mono- and polymorphonuclear leukocytes, was observed in the AddaVax injected muscles (FIG. 10A, v and vi, arrows). Ten percent DMSO in PBS was injected into the vehicle control animals (FIG. 10A, vii). Increased cellular infiltration in AddaVax-injected muscle was mirrored by higher local expression of the proinflammatory cytokine IL-6, KC, and MCP-1 (FIG. 10B). Furthermore, increased local expression of IL-6 and KC correlated with increased circulating levels of IL-6 and KC in sera (FIG. 10C). These results indicate that 1Z105 and 1V270, alone or in combination, induce minimal inflammation both at the site of injection and systemically in comparison to AddaVax.

### 1V270 Increases Chemokine Expression in Muscles at the Site of Injection

Antigen presenting cells migrate by sensing the gradients of chemokine concentrations. Hence, the chemokine expression induced by injection of 1Z105 and 1V270 were evaluated by RT-qPCR methods. 1V270 induced significantly higher MIP1a (CCL3), MCP-1(CCL2) and RANTES (CCL5).

### 1Z105 Activates Human Umbilical Vein Cells and Myeloid Dendritic Cells

TLR7 expression in humans is limited to plasmacytoid DC and B cells, and different from the expression in mice. Conversely, TLR4 is abundantly expressed in different cell types in humans and mice. Thus, experiments were performed to determine whether 1Z105 and 1V270 could stimulate human umbilical vein cells (HUVEC) and myeloid dendritic cells. 1Z105, a TLR4 stimulator, induced the chemokines MCP-1, and IL-8 in HUVEC, as well as in myeloid DC, whereas TLR7 ligand 1V270 showed minimal effects on human HUVEC and mDC.

### Combined TLR4 and TLR7 Agonists Provide Enhanced Adjuvant Effects In Vitro

To evaluate whether 1Z105 can enhance the proinflammatory cytokine induction by 1V270, BMDC (C57BL/6) were incubated with 1V270 (0.025, or 0.1 μM) in the presence or absence of 1Z105 (5 μM). The levels of IL-6 and IP-10 in the culture supernatant showed that 1Z105 significantly enhanced IL-6 and IP-10 release induced by 1V270. Antigen presenting function was evaluated using splenocytes prepared from OVA MHC class II-restricted alpha beta T cell receptor (TCR) transgenic mice OT-II. Splenocytes were cultured with OVA peptide (OVA₃₂₃₋₃₃₉) for 3 days with 1Z105, or 1V270 alone or in combination. The frequency of intracellular (IC) IFNγ expressing CD4 T cells was determined by flow cytometric assay. Treatment with 1V270 alone increased intracellular IFNγ (3.7 and 6.8% by 25 and 100 nM, respectively). TLR4 ligands alone also increased intracellular IFNγ positive cells (1.7 and 2.8%, respectively). 1Z105 additively increased frequency of IFNγ-producing cells.

### 1Z105 and 1V270 Enhance Cell Mediated Immune Responses Against Influenza HA Alone or in Combination

The disclosure demonstrates that 1Z105 and 1V270 alone induced protective response to homologous, heterologous, and heterosubtypic challenge models compared to non-adjuvanted protein antigen alone. When mice were immunized three times with different chimeric HA containing HA1 conserved stalk segments, 1V270 alone or combination with 1Z105 induced stalk mediated protection against virus challenge accompanied by significantly higher antibody titers against conserved stalk segments of HA. To further evaluate whether the combined adjuvant can raise cell mediated immune responses against the stalk segment of HA, Balb/c mice were twice immunized with recombinant PR8 HA two weeks apart, and antigen specific T cell response were evaluated by IFNγ ELlspot or ELISA 6 weeks after the first immunization. The PR8 peptide array (BEI Resources, NIH) were combined to 4 pools. Pools 1-2 and 3-4 contain the head and stalk segments of HA, respectively. 1Z105 and 1V270 alone or in combination highly induced T cell response against the head segment (pool 1-2) of HA. 1V270 or 1V270/1Z105 combination adjuvant also induced stalk-specific (pool 3-4) T cell responses.

### 1Z105 Enhances Immunostimulatory Effects of 1V270 in Human PBMC

To confirm the activities of 1Z105/1V270 combined adjuvant in human cells, PBMC were stimulated with these agonists. IL-8 release by PBMC was significantly induced by the combination of 1Z105 and 1V270, whereas 1Z105 or 1V270 alone induced minimal levels of IL-8. These data indicate that the combined adjuvant is effective in human cells.

### Combined Adjuvant Prolongs Antigen Retention in B Cells in the Draining Lymph Nodes

In the mouse models of influenza infection, the combined adjuvant promoted a rapid and sustained antibody induction. To study B cell dynamics in the draining lymph nodes after injection of the combined adjuvant, mice were intramuscularly vaccinated with 1) antigen alone (Alexa Fluor 488-OVA), 2) antigen plus the combined adjuvant, and 3) the combined adjuvant alone. 3 and 24 h after injection, the draining lymph nodes were collected. 3 h after the injection antigen was delivered to B cells at similar magnitudes for the adjuvanted and non-adjuvanted immunization. However, administration of adjuvanted antigen provided sustained antigen bearing B cell population 24 h after immunization. Increased B cell number was elevated without antigen 24 h after administration.

### Combined Adjuvant Promotes Antigen Delivery by Dendritic Cells to the Draining Lymph Nodes

The combined adjuvant effectively induced influenza virus HA antigen specific helper T cell responses after one immunization. Antigen presentation to Th cells is mostly mediated by antigen presenting cells. The combined adjuvant was thus examined to determine whether it increased DC mediated antigen transportation to the draining lymph nodes from the site of injection. B6 mice were intramuscularly injected with 1) antigen alone (Alexa Fluor 488-OVA), 2) Antigen plus the combined adjuvant, and 3) the combined adjuvant alone. Increased number of antigen containing DC were accumulated in the draining lymph nodes after injection of antigen with the combined adjuvant 24 h after immunization. In addition, B-cell proliferation was enhanced by combining the two agonists.

### The Combination of 1V270 and 1Z105 Enhances CD4+ or CD8+T Cell Proliferation

Capability for induction of antigen specific CD8 T cells is one of the ideal features for adjuvants for cancer or herpes simplex virus infection. Thus, 1V270 and 1Z105 were examined to determine whether the agonists can directly induce T cell proliferation. Purified CD4+ or CD8+ T cells were incubated with 1Z105, 1V270 alone or in combination. In the presence of CD3 ligation, 1Z105 and 1V270 alone enhanced purified CD4+ or CD8+T cell proliferation. 1Z105 and 1V270 in combination enhanced induction of both CD4+ and CD8+ T cell proliferation.

The disclosure demonstrates that used in combination, the two synthetic TLR adjuvants, 1Z105 and 1V270, rapidly induce a balanced Th1- and Th2-type response that is protective against homologous and heterologous influenza viruses. Combining 1Z105 with 1V270 in different ratios during the formulation process is a potential means to modulate the Th1/Th2 ratio induced, to refine efficacy and safety, and to reduce the amount of adjuvant administered to induce a protective response. In addition to generating neutralizing humoral immunity, a robust cellular response was also induced by the combined adjuvant, which may play an important role in cross-protective immunity. The protective response was sustained, and evidence of robust B and T cell memory was observed more than 6 months after a single immunization. 1Z105 and 1V270 in combination also demonstrated efficacy when coupled with candidate universal influenza virus vaccine constructs, inducing broadly protective immunity to the HA stalk region. Effective adjuvants will almost certainly be critical for the realization of broadly protective influenza virus vaccines, and the combination of 1Z105 and 1V270 is a promising candidate for this application. Indeed, recent reports describe adjuvanted H5 subtype vaccines that induced broadly neutralizing stalk antibody responses in human trials.

A number of embodiments have been described. Nevertheless, it will be understood that various modifications may be made without departing from the spirit and scope of the description. Accordingly, other embodiments are within the scope of the following claims.

What is claimed is:

1. A composition comprising a first aqueous component and a second component, wherein said second component comprises a compound of Formula (I) and a compound of formula (II), or a composition comprising a compound of Formula (I) and a composition comprising a compound of Formula (II): 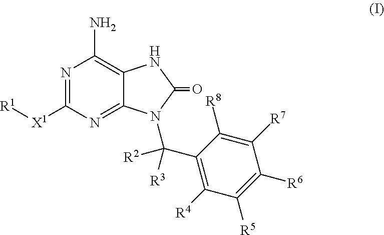
wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉)heterocyclic, substituted (C₅₋₉)heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or 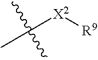
and wherein at least one of R⁴-R⁸ is 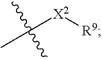
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof; 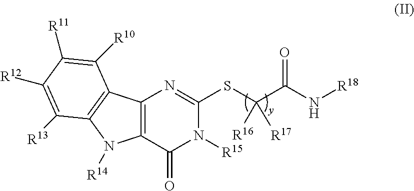
or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5; and wherein the aqueous component comprises an immunogen.

2. The composition of claim 1, wherein said immunogen is selected from the group consisting of virus, bacteria, fungus and pathogen products derived from said virus, bacteria, or fungus.

3. The composition of claim 2, wherein said virus is selected from the group consisting of influenza A virus, avian influenza virus, H5N1 influenza virus, West Nile virus, SARS virus, Marburg virus, Arenaviruses, Nipah virus, alphaviruses, filoviruses, herpes simplex virus I, herpes simplex virus II, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus.

4. The composition of claim 2, wherein said bacteria is selected from the group consisting of Bacillus cereus, Bacillus circulans and Bacillus megaterium, Bacillus anthracia, bacterial of the genus Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis.

5. The composition of claim 1, wherein the immunogen is an influenza HA stalk antigen.

6. The composition of claim 1, wherein Formula I is defined to have the structure of Formula I(a):

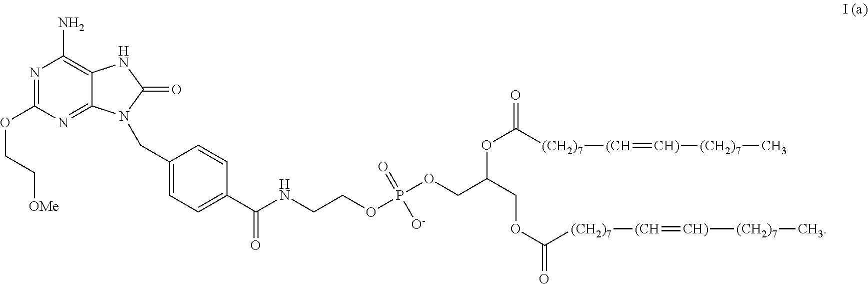

7. The composition of claim 1, wherein Formula II is defined to have the structure of Formula II(a):

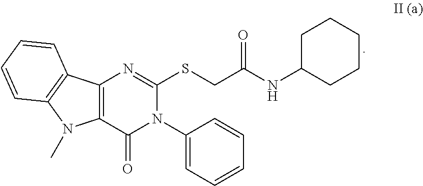 [SMILES: Cn1c2ccccc2c2nc(SCC(=O)NC3CCCCC3)n(-c3ccccc3)c(=O)c21]

8. The composition of claim 1, wherein the first aqueous component and the second component form an emulsion.

9. The composition of claim 1, wherein the first aqueous component and second component form a suspension.

10. A method to augment an immune response in a mammal comprising administering to the mammal an effective amount of a composition of claim 1.

11. A method to augment an immune response in a mammal, comprising administering to the mammal an effective amount of a composition comprising a compound of Formula (I) and a compound of formula (II), or a composition comprising a compound of Formula (I) and a composition comprising a compound of Formula (II): 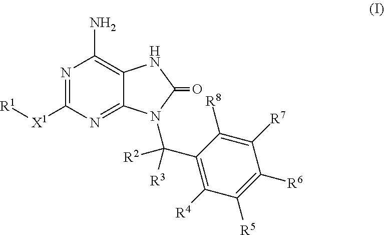
wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉) heterocyclic, substituted (C₅₋₉) heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or 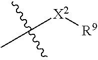
and wherein at least one of R⁴-R⁸ is 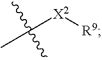
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof; 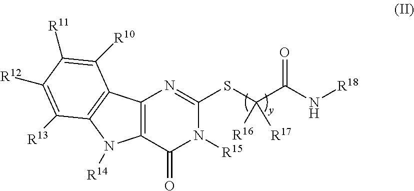
or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5.

12. The method of claim 11, wherein the composition comprising a compound of formula (I) and (II) further comprises an amount of an immunogen.

13. The method of claim 12, wherein the immunogen is a microbe, protein or a spore.

14. The method of claim 12, wherein the immunogen is an influenza HA stalk peptide.

15. The method of claim 11, further comprising administering an antigen.

16. The method of claim 15, wherein the antigen is administered concurrently with the composition.

17. The method of claim 15, wherein the antigen is administered before or after the composition.

18. The method of claim 15, wherein the antigen is a microbe, protein or spore.

19. The method of claim 15, wherein the antigen is an influenza HA stalk peptide.

20. The method of claim 12, wherein the composition is administered as a nanoemulsion.

21. The method of claim 12, wherein the composition is administered as a suspension.

22. The method of claim 11, wherein the administration is effective to prevent, inhibit or treat a microbial infection.

23. A vaccine comprising a composition comprising an antigen and an amount of a compound having Formula (I) and a compound having formula (II), or a tautomer thereof, or a pharmaceutically acceptable salt or solvate thereof: 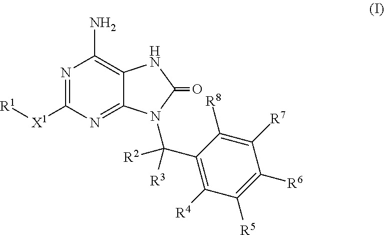
wherein X¹ is —O—, —S—, or —NR^{c}—; R¹ is hydrogen, (C₁-C₁₀)alkyl, substituted (C₁-C₁₀)alkyl, (C₆₋₁₀)aryl, or substituted (C₆₋₁₀)aryl, (C₅₋₉)heterocyclic, substituted (C₅₋₉)heterocyclic; R^{c} is hydrogen, (C₁-C₁₀)alkyl, substituted (C₁-C₁₀)alkyl, where the alkyl substituents are hydroxy, (C₃₋₆)cycloalkyl, (C₁₋₆)alkoxy, amino, cyano, or aryl; or R^{c} and R¹ taken together with the nitrogen to which they are attached form a heterocyclic ring or a substituted heterocyclic ring; R⁴-R⁸ are independently selected from a halogen, H, D, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, cyano or 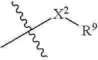
and wherein at least one of R⁴-R⁸ is 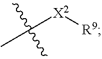
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, (C₁₋₆)alkyl, hydroxyl(C₁₋₆)alkylene, (C₁₋₆)alkoxy, (C₃-C₆)cycloalkyl, (C₁₋₆)alkoxy(C₁₋₆)alkylene, amino, cyano, halo, or aryl; X² is a bond or a linking group; and R⁹ is a phospholipid comprising one or two carboxylic esters; or a tautomer thereof; or a pharmaceutically acceptable salt or solvate thereof; 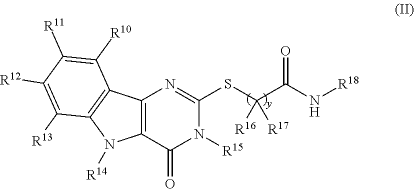
or a pharmaceutically acceptable salt thereof, wherein R¹⁰-R¹³ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is hydrogen, or substituted or unsubstituted alkyl; R¹⁵ is substituted or unsubstituted cycloalkyl, substituted or unsubstituted heterocycloalkyl, substituted or unsubstituted aryl, or substituted or unsubstituted heteroaryl; R¹⁶-R¹⁷ are independently selected from the group consisting of H, halogen, —CN, —SH, —OH, —COOH, —NH₂, —CONH₂, nitro, —CF₃, —CCI₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and y is an integer from 0 to 5.

24. The vaccine of claim 23, wherein Formula I is defined to have the structure of Formula I(a): 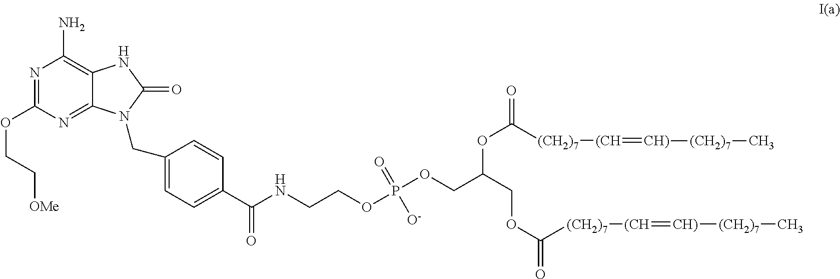
and wherein Formula II is defined to have the structure of Formula II(a): 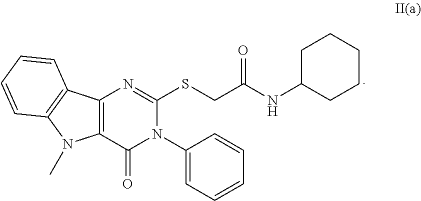 [SMILES: Cn1c2ccccc2c2nc(SCC(=O)NC3CCCCC3)n(-c3ccccc3)c(=O)c21]
